(19) United States
(12) Patent Application Publication
(10) Pub. No.: US 2025/0339178 A1
(43) Pub. Date: Nov. 6, 2025

(54) DEVICES, SYSTEMS, AND RELATED METHODS FOR ACTUATING MEDICAL DEVICES

(71) Applicant: Boston Scientific Scimed, Inc., Maple Grove, MN (US)

(72) Inventors: Kevin Jesus CERDAS ABARCA, Cartago (CR); Luis Gerardo PARRA SANCHEZ, Alajuela (CR); Fernando VALERIO GUERRERO, Alajuela (CR)

(73) Assignee: Boston Scientific Scimed, Inc., Maple Grove, MN (US)

(21) Appl. No.: 19/194,817

(22) Filed: Apr. 30, 2025

Related U.S. Application Data
(60) Provisional application No. 63/641,003, filed on May 1, 2024.

(51) Int. Cl. A61B 17/34; A61B 17/00; A61M 25/00

(52) U.S. Cl. CPC A61B 17/3478; A61B 2017/0034; A61B 2017/00367; A61M 2025/0089; A61M 25/0097

(57) ABSTRACT

Devices, systems, and related methods for actuating medical devices are disclosed herein. The disclosure includes a medical device handle. The medical device handle includes a housing defining a chamber. The medical device handle includes a shaft positioned at least partially within the chamber. The shaft includes a first depression and a second depression. The medical device handle further includes an actuator. The shaft is configured to be moveable within the chamber, relative to the housing, from a first configuration, in which a portion of the actuator is received within the first depression, and a second configuration, in which the portion of the actuator is received within the second depression.

### CROSS-REFERENCE TO RELATED APPLICATIONS

This application claims the benefit of priority to U.S. Provisional Application No. 63/641,003, filed on May 1, 2024, which is incorporated by reference herein in its entirety.

### TECHNICAL FIELD

Various aspects of this disclosure generally relate to devices, systems, and methods for actuating medical devices. In particular, aspects of this disclosure relate to handles for actuating a member proximally and/or distally.

### BACKGROUND

Endoscopic procedures may involve delivering a medical device into a body of a patient (e.g., via an insertion device such as an endoscope). The medical device may have a handle and an insertion portion. The handle may be utilized to manipulate one or more aspects of the medical device. For example, the handle may actuate an end effector of the medical device. Injection needles are examples of such medical devices. Injection needles may include a handle for gripping by an operator and a shaft (sheath) for insertion into a body of a subject (e.g., via an insertion device such as a scope). A lumen of the shaft may receive a needle that may be selectively advanced or retracted from a distal tip of the shaft. The needle may define a lumen through which a fluid, such as contrasting agent, saline, or another agent (e.g., a lifting agent) may be injected. The needle may have a distal opening that may be used to deliver the fluid to a treatment site within a body of the subject. The handle may include components for controlling the needle (e.g., advancing and retracting the needle). A need exists for handles for medical devices, such as injection needles.

### SUMMARY

Each of the aspects disclosed herein may include one or more of the features described in connection with any of the other disclosed aspects.

For example, the disclosure includes a handle for a medical device. The handle may include a housing defining a chamber. The handle may include a shaft positioned at least partially within the chamber. The shaft may include a first depression, and a second depression. The handle may include an actuator. The shaft may be configured to be movable within the chamber, relative to the housing, from a first configuration, in which a portion of the actuator is received within the first depression, and a second configuration, in which the portion of the actuator is received within the second depression.

The handle may include one or more of the following features. A portion of the actuator may be configured to be received within the first depression or the second depression. The actuator may include a button. The first depression may be distal to the second depression. The first depression may include a taper at a proximal end of the first depression so that extending the shaft distally relative to the handle dislodges the portion of the actuator from the first depression to transition the handle from the first configuration to the second configuration.

The handle may also include one or more of the following features. The handle may include a rod and a spring. The spring may surround the rod. The shaft may include a protrusion extending radially outward from an exterior surface of the shaft. The protrusion may define a cavity and a channel in communication with one another. The rod may extend through the channel and cavity. A first end of the spring may be received within the cavity. A first end of the rod may be received within a first support of the housing and a second end of the rod may be received within a second support of the housing. The spring may be compressible between an end of the cavity and a surface of one of the first support or the second support. Transitioning the handle from the first configuration to the second configuration may compress the spring.

The handle may further include one or more of the following features. The shaft may extend through a channel of the actuator. The channel may extend from a proximal end of the actuator to a distal end of the actuator. A first portion of the actuator may extend through a hole defined through a housing of the handle and a second portion of the actuator may be positioned within the chamber. The second portion of the actuator may be receivable within the first depression and the second depression. A needle may be fixedly coupled with a lumen of the shaft, such that movement of the shaft moves the needle. The actuator may include an arm configured to bias the actuator to a raised configuration. The handle may include a resilient member positioned between a distal portion of the shaft and a surface of the housing. A proximal end of the shaft may include an adapter for coupling a fluid source.

In another example, the disclosure includes a handle for a medical device. The handle may include a housing. The housing may define a chamber, a first aperture, and a second aperture. The handle may include a shaft positioned at least partially within the chamber. The shaft may include a cantilever. The shaft may be configured to be moveable within the chamber, relative to the housing, from a first configuration, in which a portion of the cantilever is received within the first aperture, and a second configuration, in which the portion of the cantilever is received within the second aperture.

The handle may include one or more of the following features. The handle may include an actuator configured to depress the portion of the cantilever to transition the handle from the first configuration to the second configuration. The handle may include a resilient member disposed between the shaft and the housing,

In yet another example, the disclosure includes a handle for a medical device. The handle may include a housing defining a chamber, a shaft positioned at least partially within the chamber; and a shuttle positioned within the chamber and fixed coupled to the shaft. The shuttle may include a first pair of protrusions and a second pair of protrusions. The shuttle may be configured to be moveable within the chamber, relative to the housing from a first configuration, in which the first pair of protrusions engages a first pin, to a second configuration, in which the second pair of protrusions engages a second pin.

The handle may include one or more of the following features. The first pair of protrusions may extend from a proximal end of the shuttle. The second pair of protrusions may extend from a distal end of the shuttle.

### BRIEF DESCRIPTION OF THE FIGURES

The accompanying drawings, which are incorporated in and constitute a part of this specification, illustrate various aspects of this disclosure and, together with the description, serve to explain the principles of the disclosure.

FIG. 1A depicts a perspective view of a portion of an exemplary medical device according to some of the aspects of the disclosure.

FIG. 1B depicts a cross-sectional view of the medical device of FIG. 1A in a first configuration.

FIG. 1C depicts a cross-sectional view of the medical device of FIG. 1A in a second configuration.

FIG. 2A depicts a perspective view of a portion of another exemplary medical device according to some of the aspects of the disclosure.

FIG. 2B depicts a cross-sectional view of the portion of the medical device of FIG. 2A in a first configuration.

FIG. 2C depicts a cross-sectional view of the portion of the medical device of FIG. 2A in a second configuration.

FIG. 3 depicts a partially transparent view of a portion of a further exemplary medical device according to some of the aspects of the disclosure.

FIG. 4A depicts a perspective view of a portion of another exemplary medical device according to some of the aspects of the disclosure

FIG. 4B depicts a first cross-sectional view of the portion of the medical device of FIG. 4A in a first configuration.

FIG. 4C depicts a second cross-sectional view of the portion of the medical device of FIG. 4A in a second configuration.

FIG. 5A depicts a perspective view of a portion of a further exemplary medical device according to some of the aspects of the disclosure

FIG. 5B depicts a cross-sectional view of the portion of the medical device of FIG. 5A.

FIG. 6A depicts a perspective view of a portion of another exemplary medical device according to some of the aspects of the disclosure.

FIG. 6B depicts a cross-sectional view of the medical device of FIG. 6A.

FIG. 7A depicts a perspective view of a portion of a further exemplary medical device according to some of the aspects of the disclosure

FIG. 7B depicts a cross-sectional view of the portion of the medical device of FIG. 7A.

FIG. 8A depicts a perspective view of a portion of another exemplary medical device according to some of the aspects of the disclosure

FIG. 8B depicts a side view of the portion of the medical device of FIG. 8A in a first configuration.

FIG. 8C depicts a side view of the medical device of FIG. 8A in a second configuration.

FIG. 9A depicts a perspective view of a portion of a further exemplary medical device according to some of the aspects of the disclosure

FIG. 9B depicts a side view of the portion of the medical device of FIG. 9A in a first configuration.

FIG. 9C depicts a side view of the portion of the medical device of FIG. 9A in a second configuration.

FIG. 10A depicts a perspective view of a portion of another exemplary medical device according to some of the aspects of the disclosure

FIG. 10B depicts a cross-sectional view of the portion of the medical device of FIG. 10A in a first configuration.

FIG. 10C depicts a cross-sectional view of the portion of the medical device of FIG. 10A in a second configuration.

### DETAILED DESCRIPTION OF THE FIGURES

Particular aspects of the disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body. Various figures of the disclosure include an arrow adjacent to a letter “D” to denote the distal direction and include an arrow adjacent to a letter “P” to denote the proximal direction.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

Further, relative terms such as, for example, “about,” “substantially,” “approximately,” etc., are used to indicate a possible variation of ±10% in a stated numeric value or range.

Where possible, reference numbers herein use the same final two digits (tens and one digits) to refer to correlating components. Unless otherwise specified, such elements with the same final two digits may have any of the same properties as one another.

This application disclosures various example handles of medical devices including a handle, an actuator, and a locking mechanism. The handle may include a shaft that is fixedly coupled to the needle. A sheath may extend from a distal end of the handle, and the needle may extend through the sheath. The needle and shaft may be movable relative to the sheath. Relative to the sheath, the needle may have a retracted, proximal configuration (e.g., a proximal configuration) where a distal tip of the needle is proximal to a distal end of the sheath, and an extended, distal configuration (e.g., a distal configuration) where the distal tip of the needle is distal to the distal end of the sheath. In the retracted configuration, the needle may be protected from damage which may be desired when an operator is inserting a portion of the medical device (e.g., the sheath and the needle) into a body lumen of a subject.

The needle and shaft may be configured to transition from the retracted configuration to the extended configuration by moving the shaft. In the extended configuration, the needle may be configured to pierce a tissue of the subject and/or to deliver a fluid or agent to a target within a body of a patient. After the device transitions to the extended configuration, the locking mechanism may be configured to maintain the shaft and needle in the extended configuration until the operator disengages components of the locking mechanism. The locking mechanism may include an actuator. Actuating the actuator of the locking mechanism may transition the needle and shaft from the extended configuration to the retracted configuration automatically.

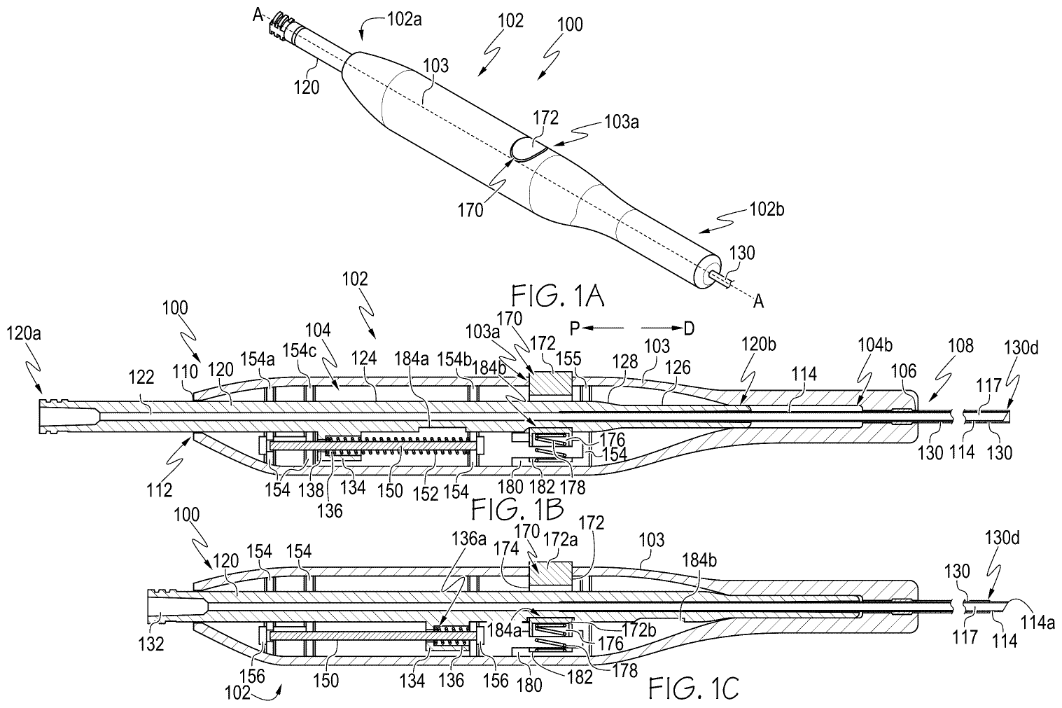

FIGS. 1A-1C depict portions of an exemplary medical device 100 based on some aspects of the disclosure. FIGS. 1A and 1B depict a first, retracted, proximal configuration of medical device 100, and FIG. 1C depicts a second, extended, distal configuration of medical device 100. Medical device 100 may include a handle 102 and a shaft or sheath 130. Sheath 130 may extend distally from a distal end of handle 102. Sheath 130 may be insertable into a body lumen of a subject (e.g., through a working channel of an insertion device such as an endoscope), and handle 102 may remain outside of the body of the subject.

Handle 102 may include a proximal end 102a and a distal end 102b. A distal portion of handle 102 may taper inwardly in diameter/width in the proximal to distal direction near distal end 102b. A proximal portion of handle 102 may taper inwardly in diameter/width in a distal to proximal direction near proximal end 102a. A housing 103 of handle 102 may include a cavity/chamber 104 configured to receive and/or contain a needle 114 and other elements, described below. Housing 103 may include a distal hole 106 defined through a distal face 108 of housing 103 at distal end 102b. Distal hole 106 may be sized and shaped so that needle 114 and sheath 130 may extend through distal hole 106. Sheath 130 may be fixedly (non-movably) coupled within distal hole 106. For example, sheath 130 may be fixedly coupled to handle 102 using adhesive, friction fit, welding, and/or other mechanisms. Housing 103 may further include a proximal hole 110 defined through a proximal face 112 of housing 103 positioned at proximal end 102a. Proximal hole 110 may be sized and shaped so that a shaft 120, discussed below, may be insertable through proximal hole 110. Medical device 100 may include a central longitudinal axis (an “axis A”) through centers of proximal hole 110 and distal hole 106.

Sheath 130 may have a distal end 130d with an element/feature configured to be operated by handle 102. For example, sheath 130 may have needle 114 at its distal end 130d. Needle 114 may be movable with respect to sheath 130, such that needle 114 may be extended from (e.g., to a position distally beyond) distal end 130d of sheath 130 (FIG. 1C) or retracted such that needle 114 is covered by sheath 130 (FIG. 1B). Needle 114 or another element (e.g., a hypotube) may extend through sheath 130 to a proximal end of sheath 130. Needle 114 (or another element) may have a lumen 117 extending therethrough. As discussed below, a portion of handle 102 may be coupled to needle 114 or another element, and needle 114 or another element may be controlled with handle 102. Although a needle 114 is described herein, it will be appreciated that medical device 100 may include other types of features at its distal tip (e.g., basket, snare, forceps, stapler, etc.), and medical device 100 is not limited by a type of instrument at its distal tip.

Handle 102 of medical device 100 may include shaft 120. Shaft 120 may include a lumen 122 extending along a central longitudinal axis of shaft 120. The central longitudinal axis of shaft 120 may be approximately coaxial with or parallel to axis A. Lumen 122 may be sized and shaped to receive at least a proximal portion of needle 114 therein. Needle 114 may be stationary (fixed) relative to shaft 120. A location of a proximal end of needle 114 shown in FIGS. 1B and 1C is merely exemplary, and needle 114 may extend any suitable length through lumen 122.

Needle 114 may be an endoscopic needle and may include lumen 117 extending from a proximal end of needle 114 to a distal end of needle 114. Needle 114 may include a sharp distal tip 114a. Lumen 117 of needle 114 may be in fluid communication with lumen 122 of shaft 120. Lumen 117 of needle 114 may be configured to receive an agent or a fluid to be delivered to a target site within a body of a patient at the distal end of needle 114, via lumen 122. Lumen 117 of needle 114 may be fluidly coupled to a fluid source and/or an agent source (e.g. a syringe or other reservoir).

Shaft 120 may include a proximal end 120a and a distal end 120b. Shaft 120 may include a proximal portion 124 and a distal portion 126. A diameter/width of distal portion 126 may be less than a diameter of proximal portion 124. Shaft 120 may include a tapered portion 128 between distal portion 126 and proximal portion 124. Proximal end 120a of shaft 120 may include a port or adapter 132 (e.g., a luer fitting, quick connect fitting, or any other suitable type of port or adapter) for coupling shaft 120 to a source of fluid (e.g., a syringe). Fluid provided through adapter 132 may flow into lumen 122 of shaft 120 and into lumen 117 of needle 114.

Proximal portion 124 may include a protrusion 134 extending radially outward of proximal portion 124. Protrusion 134 may define a cavity 136. Cavity 136 may have, for example, a cylindrical shape and may extend approximately parallel to the central longitudinal axis of shaft 120. Protrusion may further define a channel 138 at a proximal end of cavity 136. Channel 138 may be coaxial with and in fluid communication with cavity 136. Channel 138 may have a diameter that is less than a diameter of cavity 136. Cavity 136 may include a distal facing surface 136a (FIG. 1C) at a proximal end of cavity 136.

Handle 102 of medical device 100 may also include a rod 150 and a biasing member 152 (e.g., a resilient member, such as a spring) radially surrounding and coaxial with rod 150. Rod 150 and biasing member 152 may be approximately parallel with axis A. Handle 102 may include one or more internal supports 154. The one or more internal supports 154 may include a proximal support 154a, a distal support 154b, and an intermediate support 154c. Supports 154a, 154b, 154c may each include a hole or other opening (e.g., notch) sized and shaped to receive at least a portion of rod 150. Each hole of supports 154a, 154b, 154c may be a through-hole. Rod 150 may extend through each rod hole of supports 154a, 154b, 154c. Supports 154a, 154b may each include an end cap or stopper 156 (FIG. 1C). Stopper 156 of support 154a may be positioned to cover a proximal opening of the rod hole of support 154a. Stopper 156 of support 154b may be positioned to cover a distal opening of the rod hole of support 154b. Stoppers 156 may be configured to inhibit rod 150 from translating and/or rotating relative to supports 154a, 154b, 154c. In some alternative embodiments, stoppers 156 may be fixedly or removably coupled to a proximal end and distal end of rod 150, rather than to supports 154a, 154b. In an alternate embodiment without stoppers 156, the holes of supports 154a, 154b may be blind holes and the hole of support 154c may be a through hole. Rod 150 may be positioned received within and positioned between the blind holes of supports 154a, 154b so as to prevent translation and/or rotation of rod 150. In other alternative embodiments, the proximal end and distal end of rod 150 may be secured and/or fixedly attached to a structure within handle 102, such as, but not limited to, supports 154a, 154b.

Protrusion 134 may be disposed between supports 154c and 154b. Rod 150 may extend through cavity 136, channel 138, and the holes of supports 154. As mentioned above, biasing member 152 may be coaxial with rod 150 and may be positioned between distal facing surface 136a and a proximal facing surface of support 154b. Biasing member 152 may be biased to the proximal configuration of FIG. 1B.

Supports 154a, 154b, 154c may each include an opening sized and shaped to receive shaft 120. The openings of supports 154a, 154b, 154c may each include a diameter slightly larger than the diameter of proximal portion 124 (e.g., approaching approximately zero tolerance) so that shaft 120 (and, accordingly, needle 114) may move in the proximal or distal direction but rotation of shaft 120 is substantially limited. In alternatives, shaft 120 (and needle 114) may be rotatable about axis A.

Medical device 100 may have a first, proximal, retracted configuration (as seen in FIG. 1B), in which distal tip 114a of needle 114 is retracted within (covered by) distal end 130d of sheath 130. Medical device 100 may also have a second, distal, extended configuration (as shown in FIG. 1C), in which a distal tip 114a of needle 114 is extended beyond a distal end 130d of sheath 130, such that needle 114 is exposed at the distal end of device 100. Although the other embodiments disclosed herein do not depict sheath 130 or needle 114, it will be appreciated that the proximal and distal configurations of those devices may have the same configurations of sheath 130 and needle 14, discussed above. Medical device 100 may be transitioned from the proximal configuration (FIG. 1B) to the extended configuration (FIG. 1C) by moving shaft 120 distally. For example, an operator may grip proximal end 120a of shaft 120 and move shaft 120 distally.

Medical device 100 may include a locking mechanism 170 operably coupled to shaft 120 (as seen in FIGS. 1B-1C). Locking mechanism 170 may include a button 172. Button 172 may include a channel 174 (labeled in FIG. 1C) extending longitudinally through button 172 from a proximal end of button 172 to a distal end of button 172. Channel 174 may be sized and shaped to receive at least a portion of shaft 120 (e.g., at least a portion of proximal portion 124). As shown in FIG. 1A, handle 102 may define a hole 103a through housing 103 of handle 102 that is sized and shaped to receive a top (i.e., radially outer) portion 172a of button 172. Top portion 172a of button 172 may extend through hole 103a radially outward relative to axis A. Top portion 172a may extend beyond (radially outward of) or be flush with an exterior surface of housing 103. Button 172 may include a bottom portion 172b positioned within chamber 104. Bottom portion 172b may include a blind hole 176 sized and shaped to receive a first end of a biasing member 178 (e.g., a resilient member such as a spring). Housing 103 of handle 102 may include a boss or support 180, the support 180 may include a blind hole 182 sized and shaped to receive a second end of a biasing member 178 (e.g., a spring). Biasing member 178 may be configured to bias top portion 172a radially outward relative to axis A. Handle 102 may include a support 155. Support 155 may be distal to supports 154a, 154b, 154c, and may include any features of supports 154a, 154b, 154c. Support 180 may extend from an end of support 155 opposite to hole 103a.

Shaft 120 (e.g., proximal portion 124) include one or more depressions (e.g., recesses or indentations) such as a proximal depression 184a and a distal depression 184b. Depressions 184a, 184b may be sized and shaped to receive a portion of bottom portion 172b of button 172. Bottom portion 172b may be disposed within distal depression 184b in the first, retracted configuration (FIG. 1B), and bottom portion 172b may be disposed within the proximal depression 184a in the second, extended configuration (FIG. 1C). In some embodiments, distal depression 184b may include a taper at a proximal end of distal depression 184b so that shaft 120 may be extended in the distal direction to transition from the proximal, retracted configuration to the distal, extended configuration without needing to depress button 172. Depression of button 172 may compress biasing member 178, as discussed in further detail below.

With device 100 in the proximal configuration (FIG. 1B), sheath 130 may be inserted into a body lumen of a subject (e.g., using an insertion device, such as an endoscope). Distal end 130d of sheath 130 may be positioned at a target site. Resistance from biasing member 152 may prevent accidental transition of medical device 100 from the proximal configuration to the distal configuration before such a transition is desired by the operator. To expose needle 114, operator may transition shaft 120 from the proximal configuration to the distal configuration by applying a force to shaft 120 in the proximal to distal direction, parallel to axis A. For example, the operator may grip shaft 120 and move it distally. As force is being applied, bottom portion 172b of button 172 may become dislodged and/or may exit distal depression 184b by bottom portion 172b contacting and sliding along the taper of distal depression 184b, thus passively depressing button 172. Additionally or alternatively, the operator may depress button 172 by applying a radially inward force on top portion 172a. After bottom portion 172b has exited distal depression 184b, shaft 120 may move or continue to move in the distal direction as force is being applied. Because needle 114 may be fixedly coupled to shaft 120, such distal movement of shaft 120 relative to housing 103 of handle 102 may cause distal tip 114a of needle 114 to be extended distally from distal end 130d of sheath 130.

Shaft 120 may be extended distally until distal end 120b of shaft 120 reaches (e.g., abuts) a distal end 104b of chamber 104 (e.g., a proximal facing surface of housing 103) and/or bottom portion 172b of button 172 enters proximal depression 184a. Shaft 120 and needle 114 are locked in place in the distal configuration once bottom portion 172b enters proximal depression 184a. Biasing member 178 may automatically move button 172 outward. As device 100 transitions from the proximal configuration to the distal configuration, biasing member 152 may be compressed between distal facing surface 136a and support 154b. After transitioning needle 114 to the distal configuration, the operator may use distal tip 114a to pierce tissue. The operator may deliver fluid to lumen 117 from lumen 122 of shaft 120.

To retract needle 114, the operator may depress button 172 to transition needle 114 from the distal configuration to the proximal configuration. Depressing button 172 may dislodge bottom portion 172b from proximal depression 184a. Compressed biasing member 152 may automatically (passively) expand and push shaft 120 in the proximal direction until distal depression 184b reaches button 172 and bottom portion 172b enters distal depression 184b. Biasing member 178 may automatically move button 172 outward. Shaft 120 and needle 114 may be locked in the proximal configuration once bottom portion 172b enters distal depression 184b. In alternatives, biasing member 152 may be omitted, and the operator may manually move shaft 120 proximally.

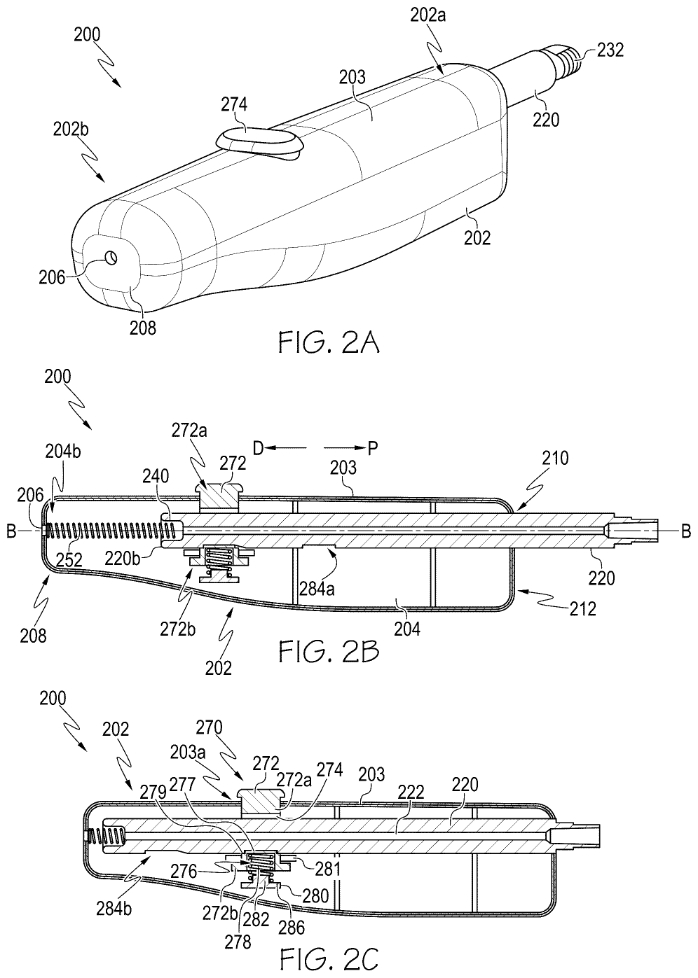

FIGS. 2A-2C depict another exemplary medical device 200 based on some aspects of the disclosure. Medical device 200 and its respective components may include any of the features of medical device 100. Where feasible, reference numbers of FIGS. 2A-2C add 100 to reference numbers of FIGS. 1A-1C to indicate correlating components. As compared with medical device 100, aspects of medical device 200 may have different components for proximal/distal biasing. The biasing mechanisms of medical devices 100, 200 are merely exemplary, and alternative structures may also be utilized. Medical device 200 may include a handle 202, a shaft 220, sheath 130 (not shown in FIGS. 2A-2C), needle 114 (not shown in FIGS. 2A-2C), and a locking mechanism 270. Sheath 130 and needle 114 are not shown in order to more clearly illustrate features of handle 202. Handle 202 may include a proximal end 202a and a distal end 202b. A housing 203 of handle 202 includes a cavity/chamber 204.

Handle 202 may include a distal hole 206 defined through a distal face 208 of housing 203 at distal end 202b. Distal hole 206 may have any of the features of hole 106. Handle 202 may further include a proximal hole 210 defined through a proximal face 212 of housing 203 and positioned at proximal end 202a. Proximal hole 210 may have any of the properties of hole 110. Medical device 200 may include a central longitudinal axis (an “axis B”) through centers of proximal hole 210 and distal hole 206.

Handle 202 of medical device 200 may include shaft 220. Shaft 220 may include a lumen 222 extending along a central longitudinal axis of shaft 220. Unless otherwise specified herein, shaft 220 may have any of the properties of shaft 120. Shaft 220 may include a cavity 240 at a distal end 220b of shaft 220. Cavity 240 may be in communication with lumen 222 of shaft 220. Handle 202 may include a biasing member 252 (e.g., a resilient member such as a coil spring). A first end of biasing member 252 may be received within cavity 240 (e.g., so that it abuts a proximal surface of cavity 240), and a second end of biasing member 252 may be fixed or coupled to (or abut) a proximal facing surface of distal end 204b of chamber 204. Biasing member 252 may radially surround and be coaxial with needle 114. In other words, needle 114 may be configured to extend through a lumen defined by windings of biasing member 252.

Medical device 200 may include a first, proximal, retracted configuration (as seen in FIG. 2B), in which needle 114 is retracted (as described above for FIG. 1B), and a second, distal, extended configuration (as seen in FIG. 2C), in which the distal end of needle 114 is extended, as discussed above for FIG. 1C.

Medical device 200 may include a locking mechanism 270 operably coupled to shaft 220 (as seen in FIGS. 2B-2C). It will be appreciated that aspects of locking mechanism 270 may also be used with device 100. Locking mechanism 270 may have any of the features of locking mechanism 170, unless otherwise specified herein. Similarly to locking mechanism 170, locking mechanism 270 may include a button 272. Button 272 may include a channel 274 (labelled in FIG. 2C) extending from a proximal end of button 272 to a distal end of button 272 and approximately coaxial with axis B. Channel 274 may be sized and shaped to receive at least a portion of shaft 220. Handle 202 may define a hole 203a through housing 203 of handle 202 that is sized and shaped to receive a portion of top portion 272a of button 272. Top portion 272a of button 272 may extend through hole 203a radially outward relative to axis B beyond an exterior surface of housing 203 for the operator to manipulate (e.g., depressing radially inward relative to axis B). Top portion 272a may include a flange that protrudes radially outward. The flange may include a diameter greater than a diameter of hole 203a. Button 272 may include a bottom portion 272b positioned within chamber 204.

Handle may include a guide support 281 with an opening or channel sized and shaped to receive bottom portion 272b. Guide support 281 may help prevent button 272 (e.g., bottom portion 272b) from rotating within chamber 204 and/or may retain button 272 and/or shaft 220 in a desired location. Bottom portion 272b of button 272 may include a blind hole 276 sized and shaped to receive a first end of a biasing member 278 (e.g., a spring). Blind hole 276 may have an open end on a side of button 272 that is opposite from top portion 272a (e.g., a bottom side in the frame of reference of FIGS. 2B and 2C). Bottom portion 272b may further include a protrusion 277 extending from a planar surface 279 toward axis B. Planar surface 279 may configured to face toward hole 203a.

Housing 203 of handle 202 may include a boss or support 280. A protrusion 282 may extend from support 280, a direction toward blind hole 276 (and top portion 272a of button 272) and approximately perpendicularly to axis B. Protrusion 282 may be approximately coaxial with blind hole 276. Protrusion 282 may include a cylindrical shape; however, such a shape is merely exemplary and protrusion 282 may have alternative (e.g., rectangular, cone, etc.) shapes. Further, protrusion 282 may have a diameter less than an inner diameter of biasing member 278, such that protrusion 282 may be received within a second end of biasing member 278 to help inhibit translation or rotation of biasing member 278 within chamber 204. The second end of biasing member 278 may contact a planar surface 286 of support 280. Depression of button 272 may compress biasing member 278. Button 272 may operate similar to or the same as button 172, described above.

Biasing member 278 may be configured to bias bottom portion 272b of button 272 radially inward relative to axis B. Shaft 220 include one or more depressions (e.g., recesses or indentations) such as a proximal depression 284a (having any of the properties of depression 184a) and a distal depression 284b (having any of the properties of depression 184b). Depressions 284a, 284b may be sized and shaped to receive a portion of bottom portion 272b of button 272. Bottom portion 272b may be disposed within distal depression 284b in the first, retracted configuration (FIG. 2B), and bottom portion 272b may be disposed within the proximal depression 284a in the second, extended configuration (FIG. 2C).

In use, medical device 200 may be used and actuated in a similar way to medical device 100. Shaft 220 may be advanced distally to transition from the proximal configuration (FIG. 2B) to the distal configuration (FIG. 2C). Button 272 may be depressed to transition from the distal configuration to the proximal configuration. Biasing member 252, similar to biasing member 152, may serve to inhibit accidental transition from the proximal configuration to the distal configuration and may automatically transition handle 202 from the distal configuration to the proximal configuration.

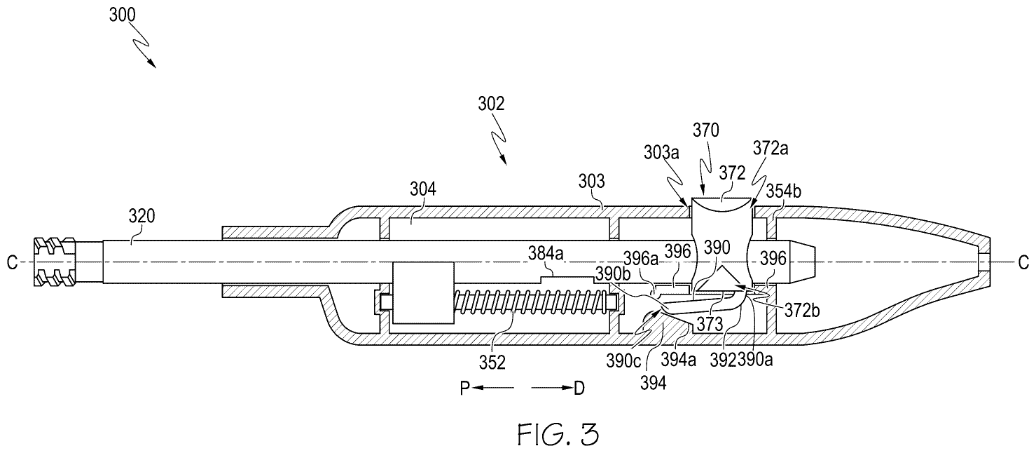

FIG. 3 depicts a portion of an exemplary medical device 300 based on some aspects of the disclosure. Medical device 300 may be substantially similar to medical devices 100 or 200, except for the differences described below. FIG. 3 depicts a view of a handle 302 of medical device 300 with a portion of a housing 303 cut away to show internal details of handle 302. Where feasible, reference numbers of FIG. 3 have the same tens and one digits as those of preceding embodiments in order to indicate correlating structures. Sheath 130 and needle 114 are not shown in order to more clearly illustrate features of handle 302.

Handle 302 of medical device 300 includes a button 372. Button 372 may include an arm 390, which may be a biasing member. Arm 390 may perform the same and/or similar functions as biasing members 178, 278 (e.g., to bias button 372 toward a hole 303a in housing 303. Button 372 may have a top portion 372a and a bottom portion 372b. Top portion 372a may be on a first side of a central longitudinal axis C of handle 302. Bottom portion 372b may be on a second, opposite side of axis C. Arm 390 may extend from a bottom surface 373 (a surface on an opposite side from top portion 372a) of bottom portion 372b. Bottom surface 373 may face a side of housing 303 that is opposite hole 303a.

Arm 390 may include a first end 390a, extending from bottom portion 372b, and a second end 390b. Second end 390b may be a free end, unconnected to another structure. Second end 390b may include a tapered or curved portion 390c. Arm 390 may include a bend 392 between first end 390a and second end 390b. Bend 392 may have an angle of about 90 degrees.

Button 372 may have a first configuration (as shown in FIG. 3) when insufficient force to depress top portion 372a of button 372 or no force is applied to top portion 372a of button 372. Button 372 may also have a second configuration in which top portion 372a is depressed (pressed radially inward). Arm 390 may be configured to bias button 372 to the first configuration. For example, arm 390 may engage with a protrusion 394 that extends inward from an inner surface of housing 303. Protrusion 394 may extend into a chamber 304 defined by housing 303. Protrusion 394 may be disposed on a portion of housing 303 that is opposite to hole 303a. A height of protrusion 394 may decrease in the proximal to distal direction, defining an angled surface 394a. Arm 390 (e.g., second end 390b) may engage with (e.g., press against) surface 394a of protrusion 394. In examples, arm 390 may be formed from a material having sufficient elasticity and rigidity to automatically transition arm 390 from the second (depressed) configuration to the first (raised) configuration by pushing upward (in a direction toward hole 303a) on protrusion 394. When arm 390 is in the raised configuration, button 372 be in the first (raised) configuration. When arm 390 is in the depressed configuration, button 372 may be in the second (depressed) configuration. Additionally or alternatively, arm 390 may be formed of a shape memory material and/or incorporate a resilient member, such as a spring.

Chamber 304 of handle 302 may include a support 396. Support 396 extend from an interior surface of chamber 304. Support 396 may extend approximately parallel to axis C. Support 396 may include a protrusion 396a, which may include a cylindrical portion. In some examples, support 396 may extend in a proximal direction from a distal support 354b. Support 396 may be configured to prevent misalignment of button 372 relative to hole 303a. Support 396 may include a hole (not shown) configured to receive a portion of button 372 and further configured to guide button 372 through holes 303a. Although not labeled in FIGS. 1A-1C, chamber 104 of handle 102 may include a support with any of the features of support 396, for example, the support of chamber 104 may similarly be configured to prevent misalignment of button 172 relative to hole 103a.

Handle 302 of medical device 300 may include a biasing member 352 (e.g., a resilient member, such as a spring), having any of the features of biasing member 152 and interacting with similar structures to biasing member 152. Alternatively or additionally, biasing member 352 may be configured similarly to biasing member 252.

In use, medical device 300 may be used and actuated in a similar way to medical devices 100 or 200. For example, medical device 100 may be transitioned from the first, proximal configuration (FIG. 3) in which needle 114 is retracted relative to sheath 130 (as described above for FIG. 1B) to the second, distal configuration in which the distal end of needle 114 is extended relative to sheath 130 (as discussed above for FIG. 1C), by moving shaft 320 distally. In the proximal configuration, a portion of bottom portion 372b of button 372 (having any of the properties of the portions of buttons 172, 272 discussed above that are received in depressions 184b, 284b) may be received within a distal depression (having any of the features of distal depression 184b, 284b) of shaft 320.

To transition from the proximal configuration to the distal configuration, an operator may grip proximal end of shaft 320 and move shaft 320 distally. Similar to distal depression 184b, 284b, a distal depression of shaft 320 (obscured by button 372 in FIG. 3) may include a taper at a proximal end of the distal depression so that shaft 320 may be extended in the distal direction to transition from the proximal, retracted configuration to the distal, extended configuration without needing to depress button 372. Shaft 320 may be extended distally until the distal end of shaft 320 reaches (e.g., abuts) the distal end of chamber 304 and/or a portion of bottom portion 372b of button 372 enter a proximal depression 384a (having any of the properties of proximal depressions 184a, 284a) of shaft 320.

During the transition between the proximal configuration and the distal configuration, top portion 372a of button 370 may move downward/radially inward, and, as a result, arm 390 may flex upwardly toward opening 303a/radially inwardly toward axis C. For example, a top/radially inward surface of arm 390 may contact and/or be adjacent to support 396. Second end 390b of arm 390 may be distal to (e.g., adjacent to) protrusion 396a. In the distal configuration, a portion of button 370 may be received within proximal depression 384a. Due to a resilience of arm 390 and a space provided by proximal depression 384a, top portion 372a of button 370 may move upward/radially outward, until a portion of button 370 is received within and engages with proximal depression 384a (in any manner described above with respect to devices 100 and 200).

To retract needle 114 and transition to the proximal configuration, the operator may depress button 372 by applying a force to top portion 372a of button 372. As button 372 is depressed, a portion of bottom portion 372b may dislodge from proximal depression 384a of shaft 320 as described above with respect to medical devices 100, 200. Arm 390 may flex at bend 392 and first end 390a may move closer to the interior surface of housing 303 opposite to hole 303a. A radially inner surface of arm 390 may abut and/or be adjacent to support 390, with second end 390b being distal to and/or adjacent to protrusion 396a. After button 372 has been sufficiently depressed such that bottom portion 372b has been dislodged from the proximal depression of shaft 320, shaft 320 and the needle 114 may automatically be moved in the proximal direction via biasing member 352.

Shaft 320 may be moved in the proximal direction by biasing member 352 until bottom portion 372b is aligned with the distal depression of shaft 320 such that a portion of bottom portion 372b may be received within the distal depression. After or before bottom portion 372b is aligned with the distal depression of shaft 320, the operator may cease applying force to top portion 372a so that arm 390 transitions from the second configuration to the first configuration (as shown in FIG. 3). As arm 390 transitions to the first configuration, second end 390b of arm 390 may push and/or bias against angled surface 394a and push top portion 372a away from protrusion 394 and push bottom portion 372b into the distal depression of shaft 320 to lock shaft 320 and needle 114 in place (e.g., in the proximal configuration).

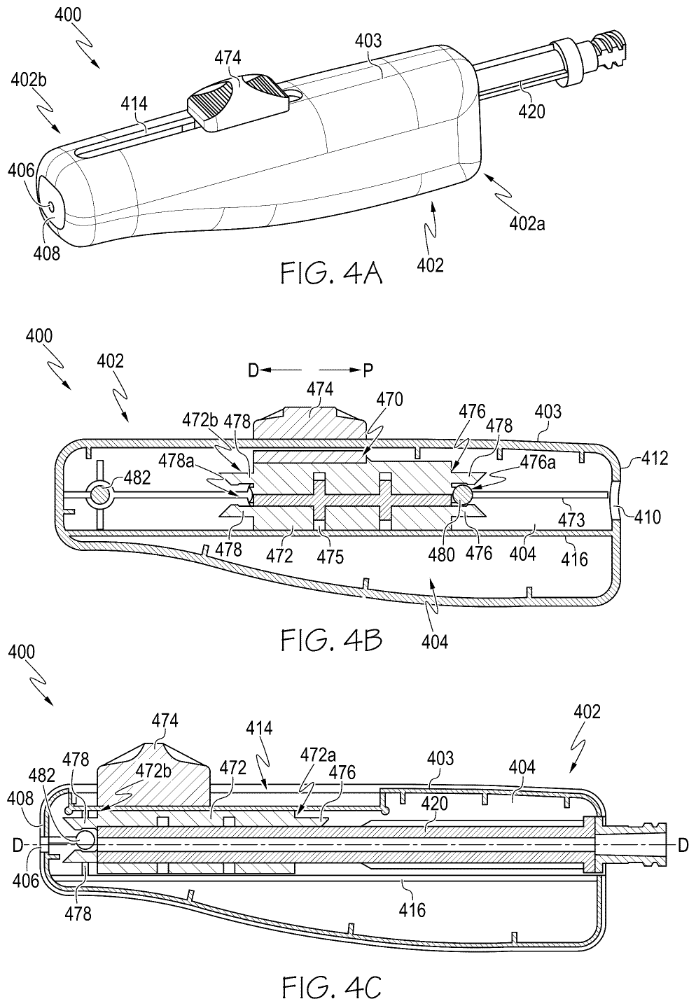

FIGS. 4A-4C depict portions of an exemplary medical device 400 based on some aspects of the disclosure. FIGS. 4A and 4B show a handle 402 of device 400 in a first, proximal, retracted configuration. FIG. 4C shows handle 402 in a second, distal, extended configuration. FIG. 4B shows a cross-section of a handle 402 of device taken along a first plane, and FIG. 4C shows a cross-section of handle 402 taken along a second plane, different from the first plane. The cross-section of FIG. 4B is further into the page than the cross-section of FIG. 4C. Where feasible, reference numbers of FIGS. 4A-4C the same tens and one digits as those of preceding embodiments in order to indicate corresponding structures. Medical device 400 may include handle 402, a shaft 420, sheath 130 (not shown), needle 114 (not shown), and a locking mechanism 470. Sheath 130 and needle 114 are not shown in order to more clearly illustrate features of handle 402.

Handle 402 may include a proximal end 402a and a distal end 402b. A distal portion of handle 402 may taper (decrease) in diameter in the proximal to distal direction near distal end 402b. Handle 402 may include a housing 403 defining a cavity/chamber 404. Handle 402 may include a distal hole 406 defined through a distal face 408 of housing 403 at distal end 402b. Handle 402 may further include a proximal hole 410 defined through a proximal face 412 of housing 403 positioned at proximal end 402a. Proximal hole 410 may have any of the properties of hole 110. Medical device 400 may include a central longitudinal axis (an “axis D”) through centers of proximal hole 410 and distal hole 406.

Handle 402 of medical device 400 may include a shaft 420. Unless otherwise specified herein, shaft 420 may have any of the properties of shaft 120, 220, 320. Medical device 400 may have a first, proximal, retracted configuration (as seen in FIG. 4B), in which shaft 420 is in a proximal configuration and distal tip 114a of needle 114 is retracted within (covered by) a distal end 130d of sheath 130. Medical device 400 may also have a second, distal, extended configuration (as shown in FIG. 4C), in which shaft 420 is in a distal configuration and distal tip 114a of needle 114 is extended beyond a distal end 130d of sheath 130, such that needle 114 is exposed at the distal end of device 400.

Medical device 400 may include a locking and actuation mechanism 470 operably coupled to shaft 420 (as seen in FIG. 4C). Locking mechanism 470 may include a shuttle 472 fixedly coupled to shaft 420 at a distal portion of shaft 420. As shown in FIG. 4B, shuttle 472 may include a lumen or cavity 475 configured to receive and/or contain a distal portion of shaft 420. Shuttle 472 may be slidable (longitudinally movable) within a chamber 404 defined by housing 403, in the proximal and/or distal directions. Medical device 400 may further include a rib or rail 473 positioned within chamber 404. Rail 473 may extend from an inner surface of housing 403 and may be parallel with axis D. Shuttle 472 may be couplable to and/or slidable along rail 473 to help guide shuttle 472 and to help maintain shaft 420 approximately coaxial with axis D when shuttle 472 is moved in the proximal or distal direction within chamber 404. Rail 473 may be configured to align or guide shuttle 472 and/or shaft 420 along axis D. Further, rail 473 may be configured to reinforce housing 403. Chamber 404 may include an internal wall 416 extending parallel with axis D from proximal end 402a to distal end 402b. Internal wall 416 may serve as a surface along which shuttle 472 slides proximally/distally and may constrain shuttle 472 to movement along a longitudinal direction.

Shuttle 472 may be coupled to a slider 474. Slider 474 may be a knob or other actuator that is disposed outside of housing 403. Slider 474 may be moveable along a slot 414 (FIG. 4A) formed in housing 403, between a distal end of slot 414 to a proximal end of slot 414. Slider 474 may be attached to and/or extend radially outward from shaft 420 (e.g., slider 474 may be an element of shaft 420). In one or more embodiments, instead of or in additional to being attached shaft 420, slider 474 may be attached (e.g., fixedly coupled) to shuttle 472 so that shuttle 472 moves with slider 474. An operator may contact or otherwise manipulate and/or move slider 474 to a desired position. It should be understood that when slider 474 is positioned at a distal end of slot 414, medical device 400 may be in the distal configuration (FIG. 4C), and when slider 474 is positioned at the proximal end of the slot, medical device 400 may be positioned in the proximal configuration (FIG. 4C). In the distal configuration, as discussed with respect to device 100, needle 114 may be exposed while, in the proximal configuration, needle 114 may be retracted.

Shuttle 472 includes a proximal end 472a and a distal end 472b. As shown particularly in FIG. 4B, Proximal end 472a may include a first pair of protrusions 476. Distal end 472b may include a second pair of protrusions 478. Each protrusion of first pair of protrusions 476 may extend from proximal end 472a in the proximal direction. Similarly, each protrusion of the second pair of protrusions 478 may extend from distal end 472b in the distal direction. A notch 476a may be formed between first pair of protrusions 476. A notch 478a may be formed between second pair of protrusions 478.

Handle 402 (e.g., housing 403) may include a proximal post or pin 480 and a distal post or pin 482. Pins 480, 482 may be positioned along (e.g., on) axis D. Shuttle 472 may be positioned longitudinally between pins 480, 482. Distal pin 482 may be configured to snap-fit (e.g., couple) with second pair of protrusions 478 when a sufficient force is applied to slider 474 in the distal direction so that distal pin 482 is received in notch 478a. Similarly, proximal pin 480 may be configured to snap-fit with first pair of protrusions 476 when a sufficient force is applied to slider 474 in the proximal direction so that proximal pin 480 is received in a space within notch 476a. It should be understood that each pair of protrusions 476, 478 may be configured to flex radially outward relative to axis D as the respective pin 480, 482 is being received within respective notch 476a, 478a, and then flex radially inward after the respective pin 480, 482 has been received within respective notch 476a, 478a.

When shuttle 472 is snap-fit to one of pins 480, 482; shuttle 472, shaft 420 and needle 114 will remain stationary relative to handle 402 until sufficient force is applied to slider 474 in the appropriate direction to decouple shuttle 472 from the respective pin 480, 482. For example, to disengage pin 480 from notch 476a, slider 474 may be moved distally, and to disengage pin 482 from notch 478a, slider 474 may be moved proximally.

With medical device 400 in the proximal configuration (FIG. 4B), protrusions 476 may engage pin 480 (pin 480 may be within notch 476a) and needle 114 may be retracted. To transition medical device 400 from the proximal configuration to the distal configuration in order to expose needle 114 relative to the sheath 130, the operator may apply sufficient force to slider 474 such that proximal pin 480 is decoupled from notch 476a of first pair of protrusions 476. After decoupling proximal pin 480 from first pair of protrusions 476, the operator may move slider 474 from the proximal end of slot 414 to the distal end of slot 414. As the operator moves slider 474 toward the distal end of slot 414; shuttle 472 and shaft 420 are moved in distal direction with slider 474.

After slider 474 has been moved to or near the distal end of slot 414, the operator may lock shuttle 472 (e.g., lock medical device 400 in the distal configuration) in place by applying sufficient force in the distal direction to slider 474 such that second pair of protrusions 478 and distal pin 482 are coupled together via snap-fit (e.g., pin 482 is received within notch 478a). Handle 402 may be in the distal configuration (FIG. 4C). Needle 114 may be exposed in the distal configuration.

To transition from the distal configuration (FIG. 4C) to the proximal configuration (FIG. 4B), the operator may apply sufficient force in the proximal direction to slider 474 such that distal pin 482 is decoupled from notch 478a of second pair of protrusions 478. After decoupling pin 482 from second pair of protrusions 478, slider 474 may be moved to the proximal end of slot 414. The operator may lock shuttle 472 (e.g., lock medical device 400 in the proximal configuration) in place by applying sufficient force in the proximal direction to slider 474 such that first pair of protrusions 476 and proximal pin 480 are coupled together via snap-fit (e.g., pin 480 is received within notch 476a).

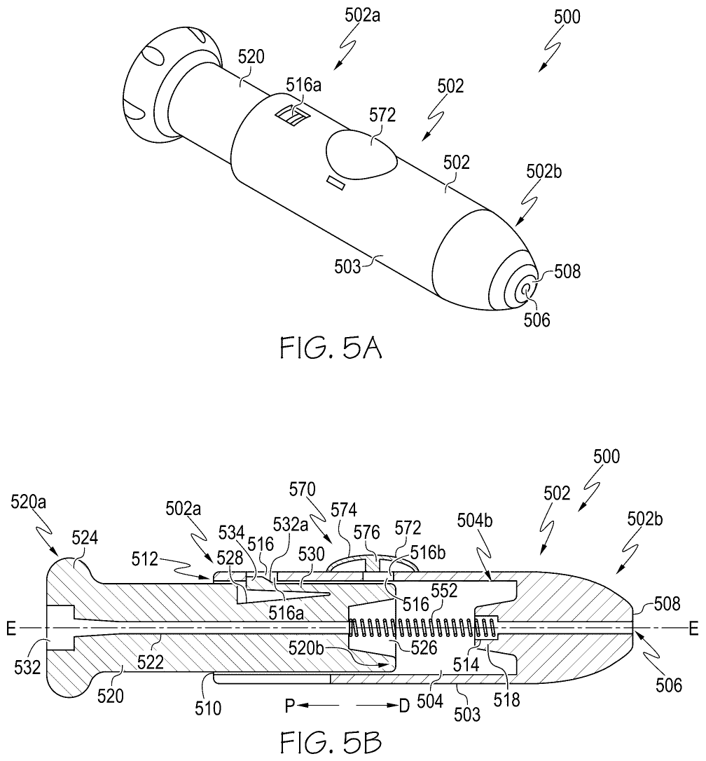

FIGS. 5A-5B depict portions of an exemplary medical device 500 based on some aspects of the disclosure. FIGS. 5A-5B show a handle 502 of device 500 in a first, proximal, retracted configuration. Device 500 also includes a second, distal, extended configuration not shown in the Figures. Medical device 500 may include handle 502, which may include a plunger or shaft 520 and a locking mechanism 570.

Handle 502 may include a proximal end 502a and a distal end 502b. Handle 502 may have a generally cylindrical shape. A distal portion of handle 502 may taper inwardly in diameter/dimension in the proximal to distal direction. A housing 503 of handle 502 may include a cavity/chamber 504. Chamber 504 may include a generally cylindrical shape. Handle 502 may include a distal hole 506 defined through a distal face 508 of housing 503 at distal end 502b. Distal hole 506 may be sized and shaped so that sheath 130 (and needle 114 extending through sheath 130) may extend through distal hole 506. For ease of illustrating the features of handle 502, sheath 130 and needle 114 are not shown in FIGS. 5A and 5B. Sheath 130 may be fixedly (non-movably) coupled within distal hole 506 and/or a channel 514 extending through a distal portion of housing 503, described in further detail below.

Handle 502 may further include a proximal hole 510 defined through a proximal face 512 of housing 503 positioned at proximal end 502a. Proximal hole 510 may be sized and shaped so that shaft 520, discussed below, may be insertable through proximal hole 510. As compared with proximal holes of the aspects disclosed above, proximal hole 510 may have a greater width/diameter to accommodate a greater width/diameter of shaft 520. Medical device 500 may include a central longitudinal axis (an “axis E”) through centers of proximal hole 510 and distal hole 506.

Handle 502 of medical device 500 may include shaft 520. Shaft 520 may have a generally cylindrical shape complementary to chamber 504. Shaft 520 may include a lumen 522 extending along a central longitudinal axis of shaft 520 and having any of the properties of lumen 122. The central longitudinal axis of shaft 520 may be approximately coaxial with or parallel to axis E. Lumen 522 may be sized and shaped to receive at least a proximal portion of needle 114 therein. Lumen 117 of needle 114 (described and shown above with respect to FIGS. 1A-1C) may be in fluid communication with lumen 522 of shaft 520. Lumen 117 of needle 114 may be configured to receive an agent or a fluid to be delivered to a target site within a body of a patient at a distal end of needle 114, via lumen 522. Needle 114 may be stationary (fixed) relative to shaft 520 so that needle 114 and shaft 520 move as a unit. Proximal end 520a may include a flange 524 extending radially outward relative to axis E. Flange 524 may assist the operator in manipulating shaft 520 and handle 502.

Shaft 520 may include an adapter 532 at a proximal end 520a of shaft 520. Whereas adapter 132 is shown as being on an outer surface of shaft 120, adapter 532 may be recessed within proximal end 520a of shaft 520. Adapter 532 may be any of the types of adapters described above for adapter 132 or may be an alternative type of adapter. Alternatively, adapter 532 may protrude from or otherwise be on an external surface of shaft 520 and may have any of the features of adapters 132, 232.

Shaft 520 may interact with a biasing member 552. Biasing member 552 may serve a similar function to biasing member 252 in that biasing member 552 is configured to contact and/or otherwise interact with a distal surface of shaft 520. A first, proximal end of biasing member 552 may be received and fixed within a cavity 526 positioned at distal end 520b of shaft 520. Cavity 526 may include a truncated cone shape with the smaller diameter of the truncated cone shape being positioned proximal to the larger diameter of the truncated cone shape.

Housing 503 of handle 502 may include a proximally extending protrusion 518 at a distal end 504b of chamber 504. Protrusion 518 may be coaxial with axis E. Protrusion 518 may include a truncated cone shape with the smaller diameter of the truncated cone shape being positioned proximal to the larger diameter of the truncated cone shape. Protrusion 518 may have complementary shape to cavity 526 and may be received within cavity 526 when handle 502 is in the distal configuration (not shown). The shapes of protrusion 518 and cavity 526 are not limited to truncated cone shapes as such shape is merely exemplary and cavity 526 and protrusion 518 may have alternative (e.g., rectangular, cone, etc.) shapes. Protrusion 518 may include channel 514 extending distally from a proximal surface of protrusion 518. Channel 514 may be in fluid communication with distal hole 506. Channel 514 may be sized and shaped to receive a portion of biasing member 552. A second, distal end of biasing member 552 may be received and fixed within channel 514. Biasing member 552 may be biased to the proximal configuration of handle 502, as shown in FIG. 5B.

As mentioned above, medical device 500 may include a proximal configuration (as seen in FIG. 5B), in which shaft 520 has a proximal arrangement within chamber 504, and a distal configuration (not shown), in which shaft 520 has a distal arrangement within chamber 504.

Medical device 500 may include a locking mechanism 570. Locking mechanism 570 may include an actuator 572, such as a button. Actuator 572 may include a dome 574. Dome 574 may include dome-like, semi-spherical shape, or semi-ellipsoidal shape. Dome 574 may be positioned on an exterior surface of a housing 503 of handle 502. Actuator 572 may include a protrusion 576 extending toward axis E from an apex of dome 574. Depressing actuator 572 may extend protrusion 576 toward axis E.

Handle 502 may define one or more apertures 516. One or more apertures 516 may include a proximal aperture 516a and a distal aperture 516b. Dome 574 may be positioned over distal aperture 516b be so that when dome 574 is depressed, protrusion 576 extends through distal aperture 516b (protrusion 576 may be longitudinally and radially aligned with distal aperture 516b). Dome 574 may be formed from an elastic and/or flexible material so that dome 574 may return to an undepressed state after a depressive force as cease being applied to dome 574. Protrusion 576 may be formed from a more rigid material that dome 574. Alternatively, protrusion 576 and dome 574 may be formed form the same material.

Shaft 520 may further define an angled recess 528. A radial depth of recess 528 may increase in the distal to proximal direction. Shaft 520 may include a cantilever 530 extending proximally from a surface of shaft 520 at a proximal end of recess 528. Shaft 520 and recess 528 may at least partially longitudinally overlap. Cantilever 530 may include a protrusion 534 positioned at a proximal end of cantilever 530. Cantilever 530 may be configured to flex radially inward toward axis E. A portion of protrusion 534 may be received by either proximal aperture 516a (in the proximal configuration of handle 502) or distal aperture 516b (in the distal configuration of handle 502). Protrusion 534 may include a tapered portion 532a at a distal end of protrusion 534.

To transition handle 502 to the distal configuration, an operator may apply a distally-directed force to shaft 520 so that shaft 520 slides distally within chamber 504. For example, the operator may grip shaft 120 and move it distally. As the operator applies force to shaft 520, tapered portion 532a of protrusion 534 may contact a distal end of proximal aperture 516a and causing cantilever 530 to flex radially inward within recess 528 until a portion of protrusion 534 is no longer extended through proximal aperture 516a. As shaft 520 is extended distally within chamber 504, biasing member 552 may be compressed between shaft 520 and distal end 504b of chamber 504. Similar to biasing member 152, biasing member 552 may prevent accidental transition of medical device 500 from the proximal configuration to the distal configuration.

Cantilever 530 may remain flexed until shaft 520 has extended sufficiently within chamber 504 so that a portion of protrusion 534 may extend through distal aperture 516b. Once protrusion 534 of cantilever 530 has been received within distal aperture 516b, medical device 500 may be in the distal configuration and the operator may cease applying force to shaft 520. After cessation of force application, biasing member 552 may expand slightly and cause a proximal end of protrusion 534 to contact of proximal end of distal aperture 516b to prevent biasing member 552 from fully expending and pushing shaft 520 proximally. Moreover, once protrusion 534 has been received within distal aperture 516b, medical device 500 may be locked in the distal configuration. In the distal configuration, at least a portion of protrusion 518 may be received within cavity 526 of shaft 520.

The operator may transition medical device 500 from the distal configuration to the proximal configuration by depressing dome 574 toward axis E. Depressing dome 574 may extend protrusion 576 through distal aperture 516b and push protrusion 534 radially inward, flexing cantilever 530 radially inward toward axis E. After protrusion 534 no longer extends into distal aperture 516b, biasing member 552 may automatically (passively) expand to its neutral length and push shaft 520 proximally. Cantilever 530 may remain flexed as shaft 520 slides proximally until protrusion 534 extends into proximal aperture 516a. The proximal end of protrusion 534 may be biased against a proximal end of proximal aperture 516a via biasing member 552. The proximal end of protrusion 534 contacting the proximal end of proximal aperture 516a may prevent biasing member 552 from fully expanding and pushing shaft 520 out of proximal hole 510. After a portion of protrusion 534 has extended into proximal aperture 516a, medical device 500 may be locked in the proximal configuration. In alternatives, biasing member 552 may be omitted, and the operator may manually move shaft 520 proximally while depressing dome 574.

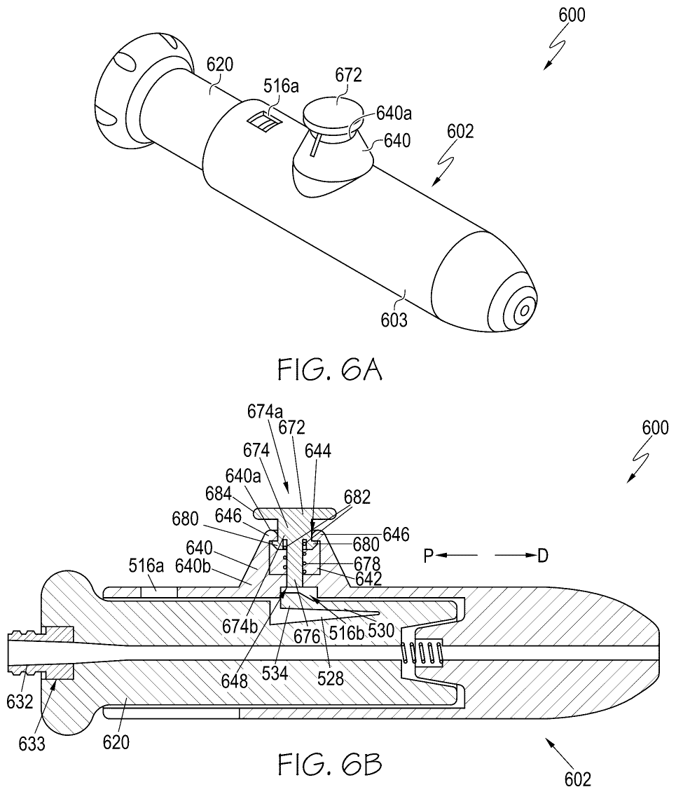

FIGS. 6A-6B depict an exemplary medical device 600 based on some aspects of the disclosure. Medical device 600 may include any of the features of medical device 500, unless specified below. FIGS. 6A-6B show a handle 602 of device 600 in a second, distal, extended configuration (e.g., distal tip 114a of needle 114 is distal to distal end 130d of sheath 130.) Device 600 also includes a first, proximal, retracted configuration not shown in the FIGS. 6A-6B but similar to the configuration of device 500 shown in FIG. 5B. Where feasible, reference numbers of FIGS. 6A-6B add 100 to the reference numbers of FIG. 5A-5B in order to indicate correlating structures.

Handle 602 of medical device 600 may include a shaft 620 including cantilever 530 and protrusion 534 of cantilever 530. Cantilever 530 may flex radially inward within recess 528 of shaft 620. Handle 602 may also include distal aperture 516b and proximal aperture 516a, described above. Shaft 620 may include a port 632. Port 632 may have any of the features of adapters 132, 532. Port 632 may protrude proximally from a proximal end of shaft 620 and may be received within a cavity 633 at a proximal end of shaft 620.

Handle 602 may include a protrusion 640 positioned on an exterior surface of a housing 603 of handle 602. Protrusion 640 may include a truncated conical shape and may decrease in diameter in a radially outward direction. Protrusion 640 may include a top portion 640a and a bottom portion 640b. Top portion 640a may include a diameter lesser than the diameter of bottom portion 640b. Protrusion 640 may define a chamber 642 that extends from top portion 640a toward bottom portion 640b. Chamber 642 may include an opening 644 at top portion 640a. Top portion 640a may include an inwardly extending flange 646 at opening 644 that decreases a diameter of opening 644 relative to radially inward portions of chamber 642. Protrusion 640 may define a channel 648. Channel 648 may extend from a radially inner end of chamber 642, which may be on an opposite side of chamber 642 from opening 644. Channel 648 may be in communication with distal aperture 516b.

Instead of actuator 572, medical device 600 may include an actuator 672 (e.g., a button). Actuator 672 may be similar to actuator 572, except as described herein. Actuator 672 may include a hub 674. Hub 674 may include a top (radially outer) side 674a and a bottom (radially inner) side 674b. Top side 674a may be positioned further from a central longitudinal axis of medical device 600. Top side 674a of hub 674 may include a disk or a radially outward extending flange 684 to help the operator in depressing actuator 672.

Actuator 672 may include a protrusion 676 (e.g., a stem) extending from bottom side 674b toward the central longitudinal axis of medical device 600. Protrusion 676 may function similarly to protrusion 576 (e.g., pushing protrusion 534 of cantilever 530 radially inward). Protrusion 676 may be sized and shaped to extend through channel 648 into distal aperture 516b.

Actuator 672 may include a biasing member 678 (e.g., a spring). Biasing member 678 may radially surround and be coaxial with protrusion 676. Actuator 672 may include one or more wings 680 (or a flange) extending from bottom side 674b and extending outward toward an inner wall of chamber 642. During assembly of medical device 600, one or more wings 680 may be configured to flex radially inward toward protrusion 676 so that wing(s) may be inserted through opening 644. After one or more wings 680 have been inserted through opening 644, one or more wings 680 may be configured to flex radially outward to prevent one or more wings 680 from exiting chamber 642 after being inserted. Actuator 672 may include a gap 682 between each of the one or more wings 680 and protrusion 676. Gap(s) 682 may be sized and shaped to receive a first end of biasing member 678. A second end of biasing member 678 may be biased against a bottom surface of chamber 642.

Similar to actuator 572, actuator 672 may be depressed by the operator. As actuator 672 is depressed, biasing member 678 may be compressed. Moreover, as actuator 672 is being depressed, protrusion 676 may extend through channel 648 into a distal aperture 516b of handle 602 to contact protrusion 534 of cantilever 530 (when protrusion 534 is disposed in distal aperture 516b and flex cantilever 530. After the operator stops depressing actuator 672, biasing member 678 may expand and push actuator 672 upward toward opening 644. Cantilever 530 may function as above to lock shaft 520 in either the proximal configuration (e.g., needle 114 retracted relative to sheath 130) or the distal configuration (e.g., needle 114 extended relative to sheath 130), as described above with respect to device 500.

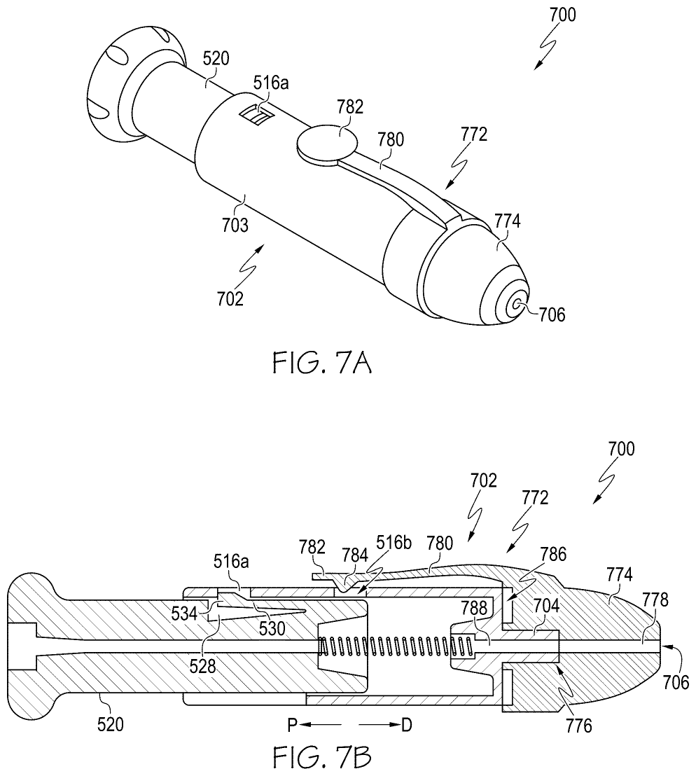

FIGS. 7A-7B depict an exemplary medical device 700 based on some aspects of the disclosure. FIGS. 7A-7B show a handle 702 of device 700 in a first, proximal, retracted configuration. Device 700 also includes a second, distal, extended configuration not shown in FIGS. 7A-7B but similar to the configuration of device 600 shown in FIG. 6B. Medical device 700 may include any of the features of medical devices 500, 600, unless otherwise specified. Where feasible, reference numbers of FIGS. 7A-7B add 100 to the reference numbers of FIG. 6A-6B and 200 to the reference numbers of FIG. 5A-5B in order to indicate correlating structures. Medical device 700 may be substantially similar to or identical medical device 500 except the differences here described. Medical device 700 may include shaft 520 including cantilever 530 and protrusion 534 of cantilever 530. Cantilever 530 may flex radially inward within recess 528 of shaft 520. Handle 702 may include proximal aperture 516a and distal aperture 516b.

Handle 702 may have any of the features of handle 502 except as described below. Handle 702 may include a cap 774 at a distal end of handle 702. Cap 774 may form a tapered portion of handle 702. Cap 774 may define a cavity 776 extending from a proximal end of cap 774 toward a distal end of cap 774. Cap 774 may include a lumen 778 extending axially therethrough. Lumen 778 may terminate in a distal hole 706. Cavity 776 and lumen 778 may be coaxial with distal hole 506 of handle 702.

Housing 703 may include a cylindrical protrusion 704 extending distally from a planar distal surface 786 of housing 703. Protrusion 704 may be coaxial with a distal hole 706 of handle 702. A lumen 788 may extend axially through protrusion 704. Lumen 788 may be coaxial with lumen 788. Lumen 778 may be sized and shaped to receive sheath 130 and needle 114. In alternative embodiments, handle 702 may include cavity 776 and cap 774 may include protrusion 704.

Instead of actuator 572, cap 774 of medical device 700 may include an actuator 772. Actuator 772 may extend proximally from cap 774. Actuator 772 may include an arm 780. Arm 780 may be flexible and may include an arcuate shape. Arm 780 may extend proximally from cap 774 (e.g., arm 780 may be integrally formed with cap 774 from a same, single piece of material) and be circumferentially/radially aligned with distal aperture 516b. A proximal end of arm 780 may include disk 782. Disk 782 may include a protrusion 784 extending radially inwardly toward distal aperture 516b from a bottom side of disk 782.

Protrusion 784 may perform similar functions to protrusions 576, 676. The operator may depress disk 782 to extend protrusion 784 radially inwardly through distal aperture 516b toward shaft 520 to contact protrusion 534 of cantilever 530 and flex cantilever 530. When the operator ceases depressing disk 782, arm 780 may be configured lift disk 782 and protrusion 784 upward/radially outwardly and away from shaft 520 and/or protrusion 534. Arm 780 may perform similar functions to arm 390 in that arm 780 biases disk 782 away from the central longitudinal axis of medical device 700 similar to how arm 390 biases top portion 372a away from the central longitudinal axis of medical device 300.

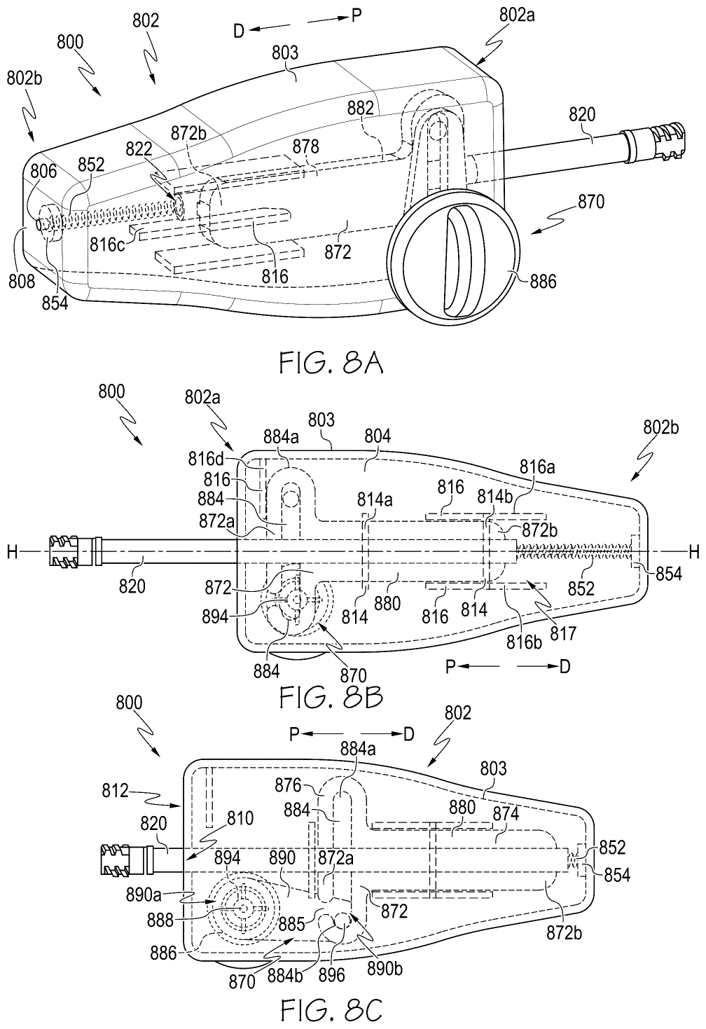

FIGS. 8A-8C depict portions of an exemplary medical device 800 based on some aspects of the disclosure. In FIGS. 8A-8C, components internal to a housing 803 of a handle 802 of device 800 are shown in broken lines. FIGS. 8A and 8B show a handle 802 of device 800 in a first, proximal, retracted configuration. FIG. 8C shows handle 802 in a second, distal, extended configuration. Medical device 800 may include handle 802, sheath 130, and needle 114. Sheath 130 and needle 114 are not shown in order to more clearly illustrate features of handle 602.

Handle 802 may include a proximal end 802a and a distal end 802b. A distal portion of handle 802 may taper (decrease) in diameter/dimension in the proximal to distal direction. A housing 803 of handle 802 may include a cavity/chamber 804. Handle 802 may include a distal hole 806 defined through a distal face 808 of housing 803 at distal end 802b. Distal hole 806 may be sized and shaped so that needle 114 and sheath 130 may extend through distal hole 806. Sheath 130 may be fixedly (non-movably) coupled within distal hole 806. Handle 802 may further include a proximal hole 810 defined through a proximal face 812 of housing 803 positioned at proximal end 802a. Proximal hole 810 may be sized and shaped so that a shaft 820 may extend through proximal hole 810. Medical device 800 may include a central longitudinal axis (an “axis H”) through centers of proximal hole 810 and distal hole 806.

Handle 802 of medical device 800 may include shaft 820, which may have any of the properties of shafts 120, 220, 320, or 420, unless specified otherwise. Shaft 820 may include a lumen 822 extending along a central longitudinal axis of shaft 820. Lumen 822 may be sized and shaped to receive at least a proximal portion of needle 114.

Handle 802 may include a biasing member 852 (e.g. a spring). A first, proximal end of biasing member 852 may be coupled to a distal end of shaft 820 and a second, distal end of biasing member 852 may be coupled to a distal end of chamber 804. Housing 803 may include a cylindrical protrusion 854 at the distal end of chamber 804 and coaxial with distal hole 806. Distal hole 806 may be defined through protrusion 854. The second end of biasing member 852 may be coupled to a proximal end of protrusion 854. Biasing member 852 may radially surround and be coaxial with needle 114. In other words, needle 114 may be configured to extend through a lumen defined by windings of biasing member 252. Biasing member 852 may be configured to bias shaft 820 to the proximal configuration of FIGS. 8A and 8B.

Chamber 804 may include one or more supports 814. One or more supports 814 may include a proximal support 814a and a distal support 814b, as shown in FIG. 8B. Each of supports 814a, 814b may define an opening sized and shaped to receive a portion of shaft 820. Each hole of supports 814a, 814b may be coaxial with axis H. The holes of supports 814a, 814b may help guide shaft 820 within chamber 804 to prevent rotation or translocation in other directions besides the proximal and distal direction. Alternatively, shaft 820 may be permitted to rotate about axis H to rotate needle 114.

Medical device 800 may include a first, proximal, retracted configuration (as seen in FIG. 8B), in which a distal end of needle 114 is retracted (as discussed with respect to device 100) and a second, distal, extended configuration (as shown in FIG. 8C), in which a distal end of needle 114 is extended (as discussed with respect to device 100).

Medical device 800 may include a locking mechanism 870 operably coupled to shaft 820 (as seen in FIGS. 8A-8C). Locking mechanism 870 may include an actuator 886 (FIG. 8A). Actuator 886 may include a knob, lever, or other suitable actuator. Actuator 886 may be a rotatable or pivotable actuator. Actuator 886 may be positioned on an exterior surface of housing 803 of handle 802 so that actuator 886 may be manipulated by the operator. Actuator 886 may include a protrusion 888 extending through a hole (not shown) defined by housing 803 into chamber 804. Locking mechanism 870 may further include a leg 890 (FIG. 8C) positioned within chamber 804. Leg 890 may include a first end 890a and second end 890b. A width of leg 890 may decrease in a direction from first end 890a to second end 890b. First end 890a may define a hole configured to receive protrusion 888. Leg 890 may be fastened at an appropriate position along protrusion 888 via one or more fasteners 894 and/or any other fixing mechanism such as, but not limited to, a snap-fit pin and/or hole attachment. Second end 890b may include a post or pin 896 extending outward from a side of leg 890.

Locking mechanism 870 may include a T-shaped member 872 within chamber 804. Member 872 may include a proximal end 872a and a distal end 872b. Member 872 include a longitudinal portion 874 (e.g., a post portion of a letter T) and a lateral portion 876 (e.g., a cross-bar portion of the letter T). In alternatives, member 872 may have other shapes (e.g., a rectangular or triangular shape). Member 872 may include a first side 878 (FIG. 8A) and a second side 880 (FIGS. 8B and 8C), and a thickness 882 in between sides 878, 880. Second side 880 may be fixedly coupled to shaft 820 and first side 878 may contact a protrusion or support extending from an interior surface of chamber 804 and may face actuator 886. In FIG. 8A, first side 878 is facing the viewer and second side 880 is facing away from the viewer. In FIGS. 8B-8C, second side 880 is facing the viewer and first side 878 is facing away from the viewer.

Lateral portion 876 may define a slot 884 approximately parallel to a longitudinal axis of lateral portion 876 and/or approximately perpendicular to axis H. Slot 884 may include a first end 884a and a second end 884b. Pin 896 of leg 890 may extend through slot 884 and be movable within slot 884 (e.g., pin 896 may move from first end 884a to second end 884b and vice versa).

Lateral portion 876 may include an opening 885 in communication with slot 884 at a proximal end of lateral portion 876. Opening 885 may be sized and shaped so that a width of opening 885 may be slightly smaller than a diameter of pin 896 so that a portion of pin 896 may be received by opening 885 but pin 896 may not exit slot 884 via opening 885. When a portion of pin 896 is received within opening 885, member 872 may be prevented from moving in the proximal direction via biasing member 852 absent the operator manually rotating actuator 886 in the appropriate direction.

Housing 803 may include one or more member supports 816 (labeled in FIG. 8B) extending from one or more interior surfaces of housing 803. Each of the supports 816 may help guide member 872 along axis H and may help prevent member 872 from moving too far in either a proximal and/or distal direction. One or more supports 816 may extend within chamber 804 parallel with axis H. One or more supports 816 may include a first support 816a and a second support 816b. Longitudinal portion 874 of member 872 may be received within a gap 817 between supports 816a, 816b. A width of gap 817 between supports 816a and 816b may be approximately the same size as or slightly larger than a lateral width of longitudinal portion 874, such that longitudinal portion 874 may extend through gap 817 and be movable in the proximal or distal direction through gap 817. A radially inward facing surface of each support 816a, 816b may contact thickness 882 of member 872 (e.g., thickness 882 of longitudinal portion 874) when member 872 is stationary or moving in the proximal or distal direction.

One or more supports 816 may further include a third support 816c (FIG. 8A). Second side 880 of member 872 may contact support 816c. One or more supports 816 may also include a fourth support 816d. Fourth support 816d may extend radially inward toward axis H and be positioned proximal to proximal end 872a of member 872 and/or proximal to a proximal end of lateral portion 876. A distally facing surface of support 816c may serve to limit how far member 872 may be displaced in the proximal direction.

The operator may transition medical device 800 from the proximal configuration (FIGS. 8A-8B) to a distal configuration (FIG. 8C) by actuating actuator 886 (e.g., rotating actuator 886 in a counter-clockwise direction in the frame of reference of FIG. 8A). As the operator is rotating actuator 886, leg 890 may rotate with actuator 886. Pin 896 may move (e.g., rotate and/or translate) from first end 884a of slot 884 to second end 884b of slot 884, and member 872 may move distally as a result of pin 896 exerting a distal force on member 872. Further, while rotating actuator 886 counter-clockwise (in the frame of reference of FIG. 8A), biasing member 852 may be compressed between the distal end of shaft 820 and protrusion 854. Like biasing member 152, biasing member 852 may prevent accidental transition of medical device 800 from the proximal configuration to the distal configuration. Once medical device 800 has reached the distal configuration, a distal end of lateral portion 876 may contact proximal ends of supports 816a, 816b, and/or 816c, preventing further distal movement of member 872.

After pin 896 has moved to second end 884b of slot 884, medical device 800 may be in the distal configuration. After medical device 800 has transitioned to the distal configuration, the operator may release actuator 886. After releasing actuator 886, biasing member 852 may expand slightly, moving pin 896 toward first end 884a and moving member 872 slightly proximally until a portion of pin 896 is received within opening 885. When pin 896 is received within opening 885, medical device 800 may remain in the distal configuration. After the portion of pin 896 has been received within opening 885, member 872 may be locked in place until the operator rotates actuator 886 in the clockwise direction (in the frame of reference of FIG. 8A) so that pin 896 dislodges from opening 885 and moves toward first end 884a, as described below.

The operator may transition medical device 800 from the distal configuration to the proximal configuration by rotating actuator 886 in the clockwise direction (in the frame of reference of FIG. 8A). As the operator rotates actuator 886, pin 896 may dislodge from opening 885, and pin 896 and biasing member 852 may push member 872 in the proximal direction until lateral portion 876 contacts support 816d and/or pin 896 reaches first end 884a of slot 884. Alternatively, after dislodging pin 896 from opening 885, the operator may release actuator 886 and allow compressed biasing member 852 to automatically (passively) expand to push shaft 820 until lateral portion 876 contacts support 816d and/or pin 896 reaches first end 884a of slot 884. Once pin 896 has reached first end 884a, medical device 800 may be in the proximal configuration. Biasing member 852 may maintain and/or lock medical device 800 in the proximal configuration.

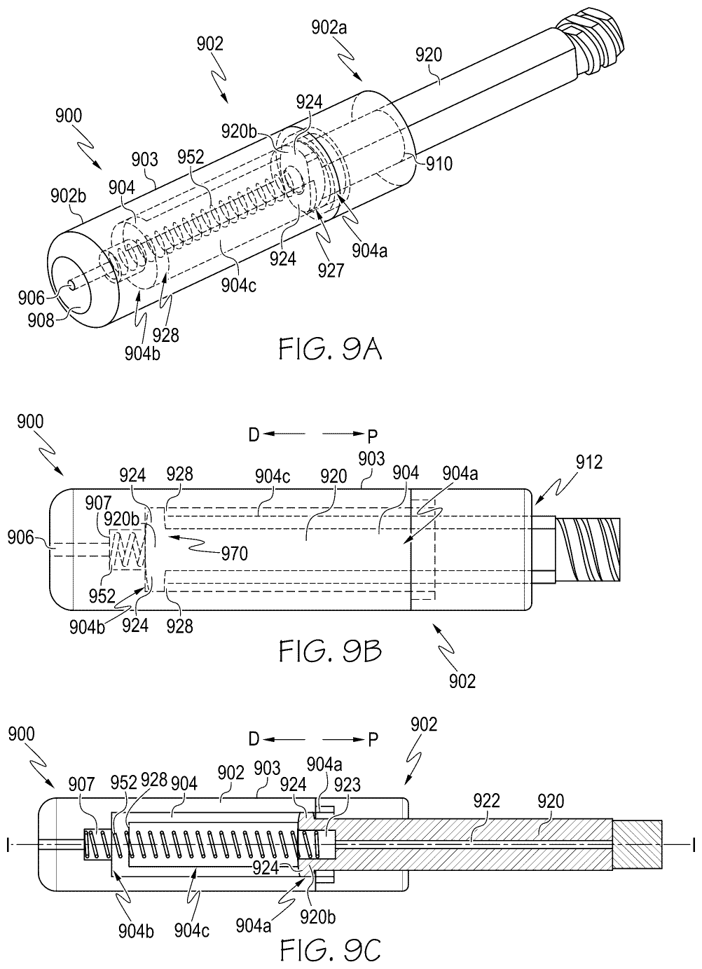

FIGS. 9A-9C depict a portion of an exemplary medical device 900 based on some aspects of the disclosure. FIGS. 9A and 9C depict medical device 900 in a first, proximal, retracted configuration. FIG. 9C depicts medical device 900 in a second, distal, extended configuration. In FIG. 9B, internal elements of medical device 900 are shown in broken lines. Medical device 900 may include a handle 902, sheath 130, and needle 114. Sheath 130 and needle 114 are not shown in order to more clearly illustrate features of handle 902.

Handle 902 may include a proximal end 902a and a distal end 902b. A housing 903 of handle 902 may include a cavity/chamber 904. Handle 902 may include a distal hole 906 defined through a distal face 908 of housing 903 at distal end 902b. Distal hole 906 may have any of the features of distal hole 106. Handle 902 may further include a proximal hole 910 defined through a proximal face 912 of housing 903 positioned at proximal end 902a. Proximal hole 910 may have any of the features of proximal hole 110. Medical device 900 may include a central longitudinal axis (an “axis I”) through centers of proximal hole 910 and distal hole 906.

Medical device 900 may include shaft 920. Shaft 920 may have any of the properties of shaft 120, except as specified herein. Shaft 920 may include a lumen 922 extending along a central longitudinal axis of shaft 920. Lumen 922 may be sized and shaped to receive at least a portion of needle 114. Needle 114 may be fixedly coupled to shaft 920 so that needle 114 and shaft 920 move as a unit. Lumen 117 of needle 114 (FIGS. 1A-1C) may be in fluid communication with lumen 922 of shaft 920.

Handle 902 may include a biasing member 952 (e.g., a resilient member such as a coil spring). Biasing member 952 may be coaxial with needle 114. Needle 114 may extend through a lumen defined by windings of biasing member 952. A first, proximal end of biasing member 952 may be received within a distal channel 923 (FIG. 9C) at distal end 920b of shaft 920. The first end of biasing member 952 may be biased against a proximal facing surface of distal channel 923. Distal channel 923 may be in communication with lumen 922. A second, distal end of biasing member 952 may be received with and biased against a distal end of a distal channel 907 of chamber 904. Distal channel 907 may be in communication with distal hole 906. Distal channel 907 and distal channel 923 may each be sized and shaped to receive an end of biasing member 952. Biasing member 952 may be configured to prevent medical device 900 from accidentally transitioning from the proximal configuration to the distal configuration by biasing handle 902 to the proximal configuration.

Distal end 920b of shaft 920 may include one or more flanges 924 extending radially outward relative to axis I. One or more flanges 924 may include two diametrically opposed flanges extending opposite one another. Distal end 920b may have a non-circular cross-sectional shape (e.g., an oblong, rectangular, rounded rectangular, or other shape), due to flanges 924. As used with respect to this embodiment, a cross-section refers to a cross-section taken perpendicularly to axis I. In alternatives, distal end 920b may include only one flange 924.

A housing 903 of handle 902 may define chamber 904. Chamber 904 may have a generally elongated shape. A middle portion 904c of chamber 904 may have a cross-sectional shape that corresponds to (keys with or mates with) a cross-sectional shape of the portion of shape of distal end 920b. A proximal portion 904a of chamber 904 may have an approximately circular cross-section (or another cross-section that achieves the function below) and may have a greater overall cross-sectional area than middle portion 904c. A surface of housing 903 may define a proximally facing shelf 927 that forms a distal surface of proximal portion 904a of chamber 904. A distal portion 904b of chamber 904 may have a same cross-sectional shape as proximal portion 904a, or a different cross-sectional shape that accomplishes the functions described below. Housing 403 may include a surface defining a distally facing shelf 928 that forms a proximal surface of distal portion 904b of chamber 904.

Medical device 900 may have a first, proximal, retracted configuration (as seen in FIG. 9A and 9C) and a second, distal, extended configuration (as seen in FIG. 9B). In the proximal configuration, distal end 920b may be received within proximal portion 904a of chamber 904. Flanges 924 may be oriented such that they are radially misaligned with middle portion 904c and flanges 924 may be aligned with and abut proximally facing shelf 927. Thus, shaft 920 may be locked in the proximal configuration due to interactions between flanges 924 and proximally facing shelf 927.

The operator may transition medical device 900 from the proximal configuration to the distal configuration by rotating shaft 920 (e.g., by approximately 90 degrees), such that flanges 924 are aligned with middle portion 904c of chamber 904 and misaligned with proximally facing shelf 927. The operator may extend shaft 920 distally through middle portion 904c of chamber 904 relative to housing 903. For example, the operator may grip shaft 920 and move it distally. As shaft 920 is extended distally through middle portion 904c of chamber 904, middle portion 904c may guide 920 and prevent or limit rotation of shaft 920 relative to axis I. As shaft 920 is extended through chamber 904, biasing member 952 may be compressed. After distal end 920b and flanges 924 have been extended to distal portion 904b of chamber 904, shaft 920 may be rotated approximately 90 degrees, clockwise or counter-clockwise, so that a proximal surface of flanges 924 aligns with shelves 928. Flanges 924 may be misaligned with middle portion 904c of chamber 904. After aligning flanges 924 and shelves 928, the operator may release shaft 920. Releasing shaft 920 may allow biasing member 952 to automatically (passively) expand and bias shaft 920 in the proximal direction so that flanges 924 press against shelves 928. The alignment of flanges 924 and shelves 928 locks shaft 920 in the distal configuration such that distal tip 114a of needle 114 is distal to distal end 130d of sheath 130.

The operator may transition medical device 900 from the distal configuration to the proximal configuration by rotating shaft 920 approximately 90 degrees, clockwise or counter-clockwise, so that flanges 924 and shelves 928 are no longer aligned. After rotating shaft 920, biasing member 952 may automatically (passively) expand and push shaft 920 proximally to proximal portion 904a of chamber 904. Alternatively, biasing member 952 may be omitted and an operator may manually move shaft 920 proximally. After distal end 920b may be received within proximal portion 904a of chamber 904, shaft 920 may be locked in place by rotating shaft 920 approximately 90 degrees so that flanges 924 may be oriented such that they are radially misaligned with middle portion 904c and flanges 924 may be aligned with and abut proximally facing shelf 927.

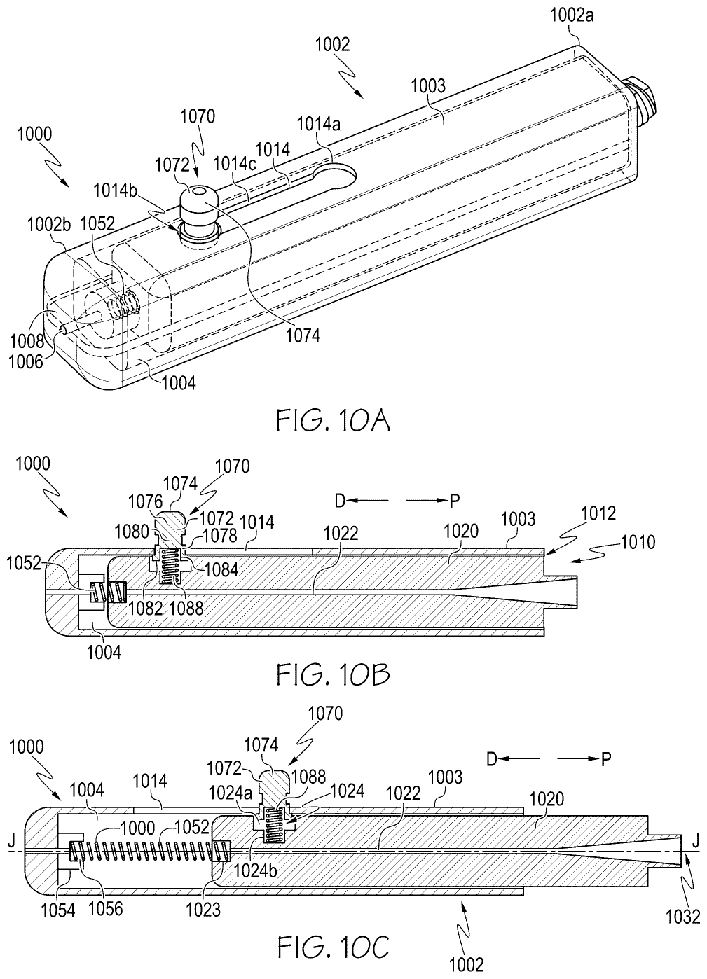

FIGS. 10A-10C depict an exemplary medical device 1000 based on some aspects of the disclosure. FIG. 10C depict a first, proximal, retracted configuration of medical device 1000 and FIGS. 10A-10B depict a second, distal, extended configuration of medical device 1000. Medical device 1000 may include a handle 1002, sheath 130, and needle 140. Sheath 130 and needle 114 are not shown in order to more clearly illustrate features of handle 1002. In FIG. 10A, internal elements are shown in broken lines.

Handle 1002 may include a proximal end 1002a and a distal end 1002b. Handle 1002 may include rectangular prism shape. A housing 1003 of handle 1002 may include a cavity/chamber 1004. Chamber 1004 may include a rectangular prism shape; however, such shape is merely exemplary and chamber 1004 may have alternative shapes (e.g., cylinder, cone) shapes. Handle 1002 may include a distal hole 1006 defined through a distal face 1008 of housing 1003 at distal end 1002b. Distal hole 1006 may have any of the features of distal hole 106. Sheath 130 may be fixedly (non-movably) coupled within distal hole 1006. Handle 1002 may further include a proximal hole 1010 defined through a proximal face 1012 of housing 1003 positioned at proximal end 1002a. Proximal hole 1010 may have a greater size with respect to proximal face 1012 than proximal hole 110 of proximal face 112 of device 100. Medical device 1000 may include a central longitudinal axis (an “axis J”) through centers of proximal hole 1010 and distal hole 1006.

Handle 1002 of medical device 1000 may include shaft 1020. Shaft 1020 may include a lumen 1022 extending along a central longitudinal axis of shaft 1020. Lumen 1022 may be sized and shaped to receive at least a portion of a needle 114. Needle 114 may be fixedly coupled to shaft 1020 so that needle 114 and shaft 1020 move as a unit. Lumen 117 of the needle 114 may be fluidly coupled to a fluid source and/or an agent source (e.g. a syringe or other reservoir).

Shaft 1020 may include a rectangular prism shape; however, such shape is merely exemplary and shaft 1020 may have alternative shapes (e.g., cylinder, cone) shapes. The shape of shaft 1020 may be complementary to the shape of chamber 1004. A proximal end of shaft 1020 may include an adapter 1032. Adapter 1032 may include any of the features of adapters 132, 232, 532.

Handle 1002 may include a biasing member 1052 (e.g., a spring). A first, proximal end of biasing member 1052 may be received within a distal channel 1023 at a distal end of shaft 1020. A second, distal end of biasing member 1052 may be biased against a distal end of chamber 1004. The distal end of chamber 1004 may include a cylindrical protrusion 1054 extending proximally from the distal end of chamber 1004 and coaxial with distal hole 1006. Cylindrical protrusion 1054 may include a distally extending channel 1056 in communication and coaxial with distal hole 1006. The second end of biasing member 1052 may be received within channel 1056 and biased against a distal end of channel 1056. Biasing member 1052 may be configured to bias shaft 1020 to the proximal configuration.

Medical device 1000 may include a locking mechanism 1070 operably coupled to shaft 1020 (as seen in FIGS. 10A-10C). Locking mechanism 1070 may include an actuator 1072. Actuator 1072 may include a button 1074 that may be depressed by the operator. At least a portion of button 1074 may be received within and slideable along a slot 1014 defined through handle 1002. Slot 1014 may include a proximal end 1014a, a distal end 1014b, and a middle portion 1014c. Proximal end 1014a and distal end 1014b may each have an approximately circular shape. Middle portion 1014c may have a width less than diameters of the circular holes of ends 1014a, 1014b.

Button 1074 may be slidable from distal end 1014b to proximal end 1014a, and vice versa. Button 1074 may include a top portion 1076, a bottom portion 1078, and a middle portion 1080 positioned between top portion 1076 and bottom portion 1078. Top portion 1076 and bottom portion 1078 may each have a width/diameter greater than the width/diameter of middle portion 1014c. Middle portion 1080 may have a diameter less than the width of middle portion 1014c of slot 1014.

Bottom portion 1078 of button 1074 may define a blind hole 1084 coaxial with the central longitudinal axis of button 1074 and approximately perpendicular to axis J. Hole 1084 may extend toward top portion 1076. Shaft 1020 may include a blind hole 1024 defined through a side of shaft 1020 facing button 1074 and extending toward axis J. Blind hole 1024 may include a top portion 1024a and a bottom portion 1024b. Bottom portion 1024b may be positioned nearer axis J than top portion 1024a. Top portion 1024a may have a diameter/width greater than a diameter/width of bottom portion 1024b.

Locking mechanism 1070 may include a biasing member 1088 (e.g., a spring). A first end of biasing member 1088 may be received within blind hole 1084 of button 1074 and a second end of biasing member 1088 may be received within top portion 1024a and bottom portion 1024b of shaft 1020. Depressing button 1074 may compress biasing member 1088 between a radially outer surface of bottom portion 1024b and a radially inner surface of blind hole 1084. A radially inward end of bottom portion 1078 of button 1074 may further include a flange 1082 extending radially outward relative to the central longitudinal axis of button 1074. Flange 1082 may have a diameter/width greater than the diameters of ends 1014a, 1014b of slot 1014 and greater than the width of middle portion 1014c of slot 1014. The diameter/width of top portion 1024a of blind hole 1024 may be greater than the diameter/width of flange 1082 such that flange 1082 may be received within top portion 1024a. The diameter of bottom portion 1024b of blind hole 1024 may be less than the diameter of flange 1082.

Medical device 1000 may include a first, proximal, retracted configuration (as seen in FIG. 10C), where distal tip 114a of needle 114 is proximal to distal end 130d of sheath 130, and a second, distal, extended configuration (as seen in FIGS. 10A-10B), where distal tip 114a of needle 114 is distal to distal end 130d of sheath 130. When medical device 1000 is in the distal configuration, button 1074 may be received within and extend through distal end 1014b of slot 1014. In the distal configuration, biasing member 1052 may be compressed and the distal end of shaft 1020 may abut a proximal end of protrusion 1054.

The operator may transition medical device 1000 from the proximal configuration to the distal configuration by depressing button 1074 until middle portion 1080 is aligned with middle portion 1014c of slot 1014 and then applying a force to button 1074 in the distal direction. As button 1074 slides along slot 1014, flange 1082 may contact a wall of top portion 1024a of hole 1024, perpendicular to axis J, and push shaft 1020 in the distal direction. As shaft 1020 moves in the distal direction, biasing member 1052 may be compressed between the proximal end of distal channel 1023 and a distal end of channel 1056. After button 1074 has reached distal end 1014b of slot 1014, the operator may cease depressing or release button 1074 so that biasing member 1088 may expand, pushing button 1074 away from shaft 1020 until bottom portion 1078 of button 1074 is aligned with middle portion 1014c of slot 1014.

The operator may transition medical device 1000 from a distal configuration to a proximal configuration by depressing button 1074 toward axis J until flange 1082 contacts a surface of top portion 1024a and/or until middle portion 1080 of button 1074 is aligned with middle portion 1014c of slot 1014. While depressing button 1074, biasing member 1088 may be compressed between button 1074 and shaft 1020. After depressing button 1074, the operator and/or biasing member 1052 may apply a force in the proximal direction to button 1074 so that button 1074 slides along slot 1014 from distal end 1014b to proximal end 1014a. Flange 1082 of button 1074 may contact the wall of top portion 1024a of hole 1024, and push shaft 1020 in the proximal direction.

After button 1074 is received with proximal end 1014a, the operator may cease depressing and/or release button 1074 so that biasing member 1088 may expand, pushing button away from shaft 1020 until flange 1082 contacts an interior surface of chamber 1004 and/or bottom portion 1078 of button 1074 is aligned with middle portion 1014c of slot 1014. Shaft 1020 and button 1074 are in a locked configuration when bottom portion 1078 is aligned with middle portion 1014c of slot 1014 as the diameter of bottom portion 1078 is greater than the width of middle portion 1014c. In the locked configuration, shaft 1020 may be retained stationary relative to a housing 1003 of handle 1002.

While principles of this disclosure are described herein with reference to illustrative examples for particular applications, it should be understood that the disclosure is not limited thereto. Those having ordinary skill in the art and access to the teachings provided herein will recognize additional modifications, applications, embodiments, and substitution of equivalents all fall within the scope of the features described herein. Accordingly, the claimed features are not to be considered as limited by the foregoing description.

What is claimed is:

1. A medical device handle comprising:
a housing defining a chamber;
a shaft positioned at least partially within the chamber, the shaft including:
a first depression, and
a second depression; and
an actuator;
wherein the shaft is configured to be moveable within the chamber, relative to the housing, from a first configuration, in which a portion of the actuator is received within the first depression, and a second configuration, in which the portion of the actuator is received within the second depression.

2. The medical device handle of claim 1, wherein a portion of the actuator is configured to be received within the first depression or the second depression, wherein the actuator includes a button.

3. The medical device handle of claim 1, wherein the first depression is distal to the second depression.

4. The medical device handle of claim 2, wherein the first depression includes a taper at a proximal end of the first depression so that extending the shaft distally relative to the handle dislodges the portion of the actuator from the first depression to transition the handle from the first configuration to the second configuration.

5. The medical device handle of claim 1, further comprising: a rod and a spring, the spring surrounding the rod.

6. The medical device handle of claim 5, wherein the shaft includes a protrusion extending radially outward from an exterior surface of the shaft, wherein the protrusion defines a cavity and a channel in communication with one another.

7. The medical device handle of claim 6, wherein the rod extends through the channel and cavity and a first end of the spring is received within the cavity.

8. The medical device handle of claim 7, wherein a first end of the rod is received within a first support of the housing and a second end of the rod is received within a second support of the housing, wherein the spring is compressible between an end of the cavity and a surface of one of the first support or the second support.

9. The medical device handle of claim 5, wherein transitioning the handle from the first configuration to the second configuration compresses the spring.

10. The medical device handle of claim 1, wherein the shaft extends through a channel of the actuator, wherein the channel extends from a proximal end of the actuator to a distal end of the actuator.

11. The medical device handle of claim 1, wherein a first portion of the actuator extends through a hole defined through a housing of the handle and a second portion of the actuator is positioned within the chamber, wherein the second portion of the actuator is receivable within the first depression and the second depression.

12. The medical device handle of claim 1, wherein a needle is fixedly coupled within a lumen of the shaft, such that movement of the shaft moves the needle.

13. The medical device handle of claim 1, wherein the actuator includes an arm configured to bias the actuator to a raised configuration.

14. The medical device handle of claim 1, further comprising a resilient member positioned between a distal portion of the shaft and a surface of the housing.

15. The medical device handle of claim 1, wherein a proximal end of the shaft includes an adapter for coupling to a fluid source.

16. A medical device handle, comprising:
a housing defining a chamber, a first aperture, and a second aperture; and
a shaft positioned at least partially within the chamber, the shaft including a cantilever;
wherein the shaft is configured to be moveable within the chamber, relative to the housing, from a first configuration, in which a portion of the cantilever is received within the first aperture, and a second configuration, in which the portion of the cantilever is received within the second aperture.

17. The medical device handle of claim 16, further comprising an actuator configured to depress the portion of the cantilever to transition the handle from the first configuration to the second configuration.

18. The medical device handle of claim 17, further comprising a resilient member disposed between the shaft and the housing.

19. A medical device handle, comprising:
a housing defining a chamber;
a shaft positioned at least partially within the chamber; and
a shuttle positioned within the chamber and fixedly coupled to the shaft, the shuttle including a first pair of protrusions and a second pair of protrusions;
wherein the shuttle is configured to be moveable within the chamber, relative to the housing from a first configuration, in which the first pair of protrusions engages a first pin, to a second configuration, in which the second pair of protrusions engages a second pin.

20. The medical device handle of claim 19, wherein the first pair of protrusions extends from a proximal end of the shuttle, and wherein the second pair of protrusions extends from a distal end of the shuttle.
